(19) United States
(12) Patent Application Publication
(10) Pub. No.: US 2026/0207702 A1
(43) Pub. Date: Jul. 23, 2026

(54) USE OF AVPR1A AGONISTS

(71) Applicants: Viage Therapeutics Inc., Palo Alto, CA (US); Kyoto University, Kyoto (JP)

(72) Inventors: Igor D. Grachev, Millstone Twp, NJ (US); Sooin Hwang, San Rafael, CA (US); Takahide Koda, Yokohama (JP); Harald K. Murk, Westfield, NJ (US); Takashi Nishida, Kyoto-shi (JP); Kousaku Ohinata, Kyoto-shi (JP); Dirk E. Smith, Bainbridge Island, WA (US); Steven August Smith, San Jose, CA (US); Kohei Kawano, Kyoto-shi (JP)

(21) Appl. No.: 19/138,619

(22) PCT Filed: Dec. 14, 2023
(86) PCT No.: PCT/US2023/084089

§ 371 Date: Jun. 12, 2025

Related U.S. Application Data
(60) Provisional application No. 63/387,418, filed on Dec. 14, 2022.
(60) Provisional application No. 63/507,754, filed on Jun. 13, 2023.

(51) Int. Cl. A61K 38/095; A61P 25/20; A61P 25/24; A61P 25/28; A61P 29/00

(52) U.S. Cl. CPC A61K 38/095; A61P 25/20; A61P 25/24; A61P 25/28; A61P 29/00

(57) ABSTRACT

AVPR1A agonists for use in treating a subject suffering from or at risk of (i) a disease or disorder of the nervous system or (ii) an inflammatory disease or disorder, or (b) improving sleep in a subject or (c) improving cognition in a subject.

### CROSS-REFERENCE TO RELATED APPLICATIONS

This application claims the benefit of and priority to U.S. Provisional Patent Application Nos. 63/387,418 (filed on Dec. 14, 2022) and 63/507,754 (filed on Jun. 13, 2023), the disclosures of each of which are incorporated by reference herein in their entirety for all purposes.

### SEQUENCE LISTING

The present specification makes reference to a Sequence listing which has been submitted electronically in XML format and is hereby incorporated by reference in its entirety. Said XML file, created on Dec. 14, 2023, is named DGS-007WO_ST26, and is 2 kb in size.

### BACKGROUND

It is estimated that 9.5% of the adult population of the United States suffers from a mood disorder (Kessler et al., 2005, Arch Gen Psychiatry 62(6):617-627). Only about one-half of those suffering from a mood disorder receive treatment, and less than 40% of those receiving treatment receive minimally adequate treatment (Wang et al., 2005, Arch Gen Psychiatry 62(6):629-640).

It is also estimated that 18.1% of the adult population of the United States suffers from an anxiety disorder (Kessler et al., 2005, Arch Gen Psychiatry 62(6):617-627). Less than 40% of those suffering from an anxiety disorder receive treatment, and less than 35% of those receiving treatment receive minimally adequate treatment (Wang et al., 2005, Arch Gen Psychiatry 62(6):629-640).

Disorders of diminished motivation occur frequently in individuals with traumatic brain injury, with estimates for the frequency varying from 5% to 67% (Marin and Wilkosz, 2005, J Head Trauma Rehabil 20(4):377-388). Diminished motivation is also often found in individuals suffering from depression, Parkinson's disease, Alzheimer's disease, and schizophrenia, as well as individuals who have suffered a stroke, and even in healthy individuals, particularly the elderly (Bonnelle et al., 2015, Journal of Physiology 109:16-26).

### SUMMARY

The peptide LSSTQAQQSY (SEQ ID NO: 1) is useful for treating mental disorders such as mood disorders, anxiety disorders, and disorders of diminished motivation. The present disclosure is based, in part, on the identification of a putative biological target of LSSTQAQQSY (SEQ ID NO: 1), arginine vasopressin receptor 1A (AVPR1A). It is believed that LSSTQAQQSY (SEQ ID NO: 1) mediates its therapeutic effects agonizing AVPR1A. It is further believed that indications amenable to treatment by LSSTQAQQSY (SEQ ID NO: 1) are similarly amenable to treatment by other AVPR1A agonists such as vasopressin, a natural AVPR1A ligand, and synthetic AVPR1A agonists including vasopressin analogues such as desmopressin, terlipressin, and felypressin.

The present disclosure is further based, in part, on new insights into the biological activities of LSSTQAQQSY (SEQ ID NO: 1). Specifically, it has been surprisingly discovered that the peptide LSSTQAQQSY (SEQ ID NO: 1) has biological activities beyond those previously reported. For example, microdialysis studies performed with the peptide LSSTQAQQSY (SEQ ID NO: 1), detailed in the Examples, demonstrated that oral administration of the peptide surprisingly affects the levels of certain neurotransmitters in the brain, including histamine and norepinephrine. Without being bound by theory, these neurotransmitters are believed to be associated with various diseases, disorders, and conditions, for example schizophrenia and neurodegenerative diseases (e.g., Parkinson's disease), and in particular negative symptoms associated with these diseases. Further, a study performed with the peptide LSSTQAQQSY (SEQ ID NO: 1) in a lipopolysaccharide-induced model of inflammation, also detailed herein, demonstrated that oral administration of the peptide surprisingly suppresses release of TNF-α, an inflammatory cytokine associated with various diseases, including rheumatoid arthritis and inflammatory bowel disease. Further still, a study performed with the peptide LSSTQAQQSY (SEQ ID NO: 1) in human subjects, also detailed in the Examples, demonstrated that oral administration of the peptide provides pro-cognitive effects. Thus, the studies described in the Examples indicate that LSSTQAQQSY (SEQ ID NO: 1) and other AVPR1A agonists can be used to treat various diseases, disorders, and conditions.

In some aspects, the disclosure provides methods for treating diseases, disorders and conditions amenable to treatment by vagus nerve stimulation and/or modulation of histamine and/or norepinephrine levels and/or modulation of TNF-α with AVPR1A agonists.

In some aspects, the disclosure provides methods of (a) treating a subject suffering from or at risk of (i) a disease or disorder of the nervous system or (ii) an inflammatory disease or disorder, or (b) improving sleep in a subject or (c) improving cognition in a subject, the methods comprising administering to the subject a therapeutically effective amount of an AVPR1A agonist.

In some aspects, the disclosure provides methods of (a) treating a subject suffering from or at risk of (i) a disease or disorder of the nervous system or (ii) an inflammatory disease or disorder, or (b) improving sleep in a subject or (c) improving cognition in a subject, the methods comprising administering to the subject an agent comprising a means for agonizing arginine vasopressin receptor 1A (AVPR1A).

Exemplary disease and disorders that can be treated with AVPR1A agonists include inflammatory diseases and disorders, schizophrenia, psychosis, neurodegenerative diseases, gastrointestinal diseases and disorders, pain, rheumatoid arthritis, migraine, headache, substance abuse, drug addiction, seizure disorders, major depressive disorder, atypical depression, major depressive episode (MDE), treatment resistant depression, depression in the presence of a neurodegenerative disease, cognitive impairment, COVID-19 related cognitive impairment and/or depression, ADHD, autism spectrum disorders, pervasive developmental disorders, atypical autism, multiple sclerosis, PTSD, and sleep disorders.

Exemplary AVPR1A agonists include vasopressin analogs, e.g., felypressin or desmopresin.

In some embodiments, provided methods are used to treat a subject suffering from or at risk of Alzheimer's or Parkinson's disease.

In some embodiments, provided methods are used to improve cognition.

In some embodiments, provided methods are used to improve cognition.

In some embodiments, provided methods are used to treat a subject suffering from or at risk of depression.

In some aspects, the disclosure provides methods of treating a subject suffering from or at risk of Alzheimer's Disease or Parkinson's Disease comprising administering to the subject a therapeutically effective amount of a vasopressin analog or a pharmaceutically acceptable salt thereof.

In some aspects, the disclosure provides methods of improving cognition in a subject comprising administering to the subject a therapeutically effective amount of a vasopressin analog or a pharmaceutically acceptable salt thereof.

In some aspects, the disclosure provides methods of improving sleep in a subject comprising administering to the subject a therapeutically effective amount of a vasopressin analog or a pharmaceutically acceptable salt thereof.

In some aspects, the disclosure provides methods of treating a subject having or at risk of having depression, the method comprising administering to the subject a therapeutically effective amount of a vasopressin analog or a pharmaceutically acceptable salt thereof.

In some embodiments, the subject is a mammal, e.g., a human.

In some embodiments, the vasopressin analog is felypressin or desmopressin.

In some embodiments, AVPR1A agonist or agent is administered in multiple doses, e.g., more than once a day, or once a day for more than one day.

In further aspects, the disclosure provides AVPR1A agonists for use in a method of treatment described herein.

In further aspects, the disclosure provides uses of AVPR1A agonists in the manufacture of medicaments for treating subjects having or at risk of developing a disease or condition disclosed herein.

Exemplary features of methods of treatment, compounds and compositions for use in a method of treatment, and uses are described in the Detailed Description and numbered embodiments 1 to 104, infra.

### BRIEF DESCRIPTION OF THE FIGURES

FIG. 1 shows that the AVPR1A/AVPR2 inhibitor conivaptan blocks LSSTQAQQSY (SEQ ID NO: 1) activity in a tail suspension test (reduced TST immobility) (Example 1).

FIG. 2 shows that low doses of the AVPR1A/AVPR2 inhibitor conivaptan blocks LSSTQAQQSY (SEQ ID NO: 1) activity in a tail suspension test (reduced TST immobility) (Example 1).

FIG. 3 shows that the AVPR2 inhibitor tolvaptan does not block LSSTQAQQSY (SEQ ID NO: 1) activity in a tail suspension test (reduced TST immobility) (Example 1).

FIG. 4 shows that the AVPR1A/B inhibitor nelivaptan blocks LSSTQAQQSY (SEQ ID NO: 1) activity in a tail suspension test (reduced TST immobility) (Example 1).

FIGS. 5A-5G show neurotransmitter levels in mouse prefrontal cortex before and after oral administration of LSSTQAQQSY (SEQ ID NO: 1) (Example 2). FIG. 5A: histamine; FIG. 5B: norepinephrine; FIG. 5C: GABA; FIG. 5D: glutamate; FIG. 5E: glycine; FIG. 5F: dopamine; FIG. 5G: serotonin.

FIGS. 6A-6G show neurotransmitter levels in mouse striatum before and after oral administration of LSSTQAQQSY (SEQ ID NO: 1) (Example 2). FIG. 6A: histamine; FIG. 6B: norepinephrine; FIG. 6C: GABA; FIG. 6D: glutamate; FIG. 6E: glycine; FIG. 6F: dopamine; FIG. 6G: serotonin.

FIG. 7 shows plasma TNF-α levels following administration of lipopolysaccharide and the peptide LSSTQAQQSY (SEQ ID NO: 1) (Example 3).

FIG. 8A depicts the study design for single ascending dose cohorts S1, S2, S3, and S4 in a Phase 1 clinical trial of the peptide LSSTQAQQSY (SEQ ID NO: 1). (See Example 4.)

FIG. 8B depicts the study design for multiple ascending dose (MAD) Cohort 1 in a Phase 1 clinical trial of the peptide LSSTQAQQSY (SEQ ID NO: 1). (See Example 4.)

FIG. 8C depicts the crossover study design for MAD cohort 2 in a Phase 1 clinical trial of the peptide LSSTQAQQSY (SEQ ID NO: 1). (See Example 4.)

FIG. 9 shows the effect of doses of 60 mg/day (MAD1), 180 mg/day (MAD2), or 540 mg/day (MAD3), versus placebo, on overall results of the PHQ-9 questionnaire (Example 4).

FIG. 10 shows the effect of doses of 60 mg/day (MAD1), 180 mg/day (MAD2), or 540 mg/day (MAD3), versus placebo, on the CogState Detection Test (DET) of psychomotor function (Example 4).

FIG. 11 shows the effect of doses of 60 mg/day (MAD1), 180 mg/day (MAD2), or 540 mg/day (MAD3), versus placebo, on the CogState Groton Maze Learning Test (GMLT) of executive function (Example 4).

FIG. 12 shows the effect of doses of 60 mg/day (MAD1), 180 mg/day (MAD2), or 540 mg/day (MAD3), versus placebo, on the CogState Identification Test (IDN) of attention (Example 4).

FIG. 13A shows composite cognition scores in the placebo group and in the peptide-treated groups in MAD Cohort 2 (the crossover study) at day 1 (pre-dosing) and day 7 (at four hours post-dosing) in a Phase 1 clinical trial. (See Example 4.)

FIG. 13B shows radar plots for the 13 subjects in MAD Cohort 2 of a Phase 1 clinical trial for the four Cogstate tests: detection test (DET), Groton Maze Learning (GMLT), Identification test (IDN), and One Card Learning test (OCL). (See Example 4.)

FIG. 13C shows test scores in the four tests in placebos (left-most bars within each pair of bars for each test) and peptide-treated subjects (right-most bars within each pair of bars for each test). (See Example 4.)

FIG. 14 depicts the theta power (top panel) and alpha power (bottom panel) in MAD Cohort 1 across multiple dose levels in a Phase 1 clinical trial. (See Example 4.)

FIG. 15 depicts the modulation of Alpha Power in eyes open state in MAD Cohort 1 across multiple dose levels in a Phase 1 clinical trial. (See Example 4.)

FIG. 16A depicts dose-dependent insomnia sleep index (ISI) values for MAD Cohort 1 (n=6 per dose, p=0.04 based on a Mann-Whitney t-test, 1-tailed (peptide treated vs. placebo groups.)) in a Phase 1 clinical trial. (See Example 4.) FIGS. 16B ad 16C depict ISI values at day 1, day 7, and day 7 at 4 hours after dosing for placebo and peptide-treated subjects in MAD Cohorts 1 and 2 respectively from a Phase 1 clinical trial. The p-values for day 1 vs. day 7 (four hours after dosing) were 0.03 for Cohort 1 (n=6), 0.1, for Cohort 2 (n=11), and 0.01 for Cohort 1+2. (See Example 4.)

FIG. 17A depicts the alpha slow wave index (ASI; alpha/(delta+theta)) in the frontal cortex plotted against the dose of peptide administered in a Phase 1 clinical trial. FIG. 17B depicts the day 7 insomnia severity score (y-axis) plotted against ASI changes in the frontal cortex (Fz eyes closed) (x-axis) in subjects treated with peptide in a Phase 1 clinical trial. (See Example 4.)

FIG. 18 shows the effect of orally administered felypressin (0.03 mg/kg, 0.1 mg/kg, and 0.3 mg/kg) and LSSTQAQQSY (SEQ ID NO: 1) (0.3 mg/kg) in the mouse tail suspension test (Example 5). Mean±SEM (n=6).

FIG. 19 shows the effect of orally administered felypressin (0.1 mg/kg), conivaptan (3 mg/kg), and felypressin (0.1 mg/kg) plus conivaptan (3 mg/kg) in the mouse tail suspension test (Example 5). Mean±SEM (n=7).

FIG. 20 shows the effect of orally administered desmopressin (0.03 mg/kg, 0.1 mg/kg, and 0.3 mg/kg) and felypressin (0.1 mg/kg) in the mouse tail suspension test (Example 6). Mean±SEM (n=6).

FIG. 21 shows the effect of orally administered desmopressin (0.1 mg/kg, 0.3 mg/kg, and 1.0 mg/kg) and felypressin (0.1 mg/kg) in the mouse tail suspension test (Example 6). Mean±SEM (n=6).

FIG. 22 depicts immobility time (indicative of stress resilience) in mice subject to the tail suspension test (TST) after receiving vehicle or 0.03 mg/kg, 0.3 mg/kg, 3 mg/kg, or 10 mg/kg of peptide (LSSTQAQQSY (SEQ ID NO: 1)).

FIG. 23A depicts immobility time in mice subject to the tail suspension test (TST) after receiving vehicle, 1 mg/kg once, or 0.3 mg/kg peptide (LSSTQAQQSY (SEQ ID NO: 1)) once, twice, or three times at two-hour intervals FIG. 23B depicts immobility time in mice subject to the tail suspension test (TST) after receiving vehicle or peptide (LSSTQAQQSY (SEQ ID NO: 1)) daily for one day or for 5 days.

### DETAILED DESCRIPTION

### Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and claim, the singular form “a,” “an,” and “the” include plural references unless the context clearly dictates otherwise. For example, the term “an agent” includes a plurality of agents, including mixtures thereof.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

As used herein, the term “AVPR1A” refers to arginine vasopressin receptor 1A. An exemplary AVPR1A. Exemplary AVPR1A sequences are available at the Uniprot database under accession number P37288.

As used herein, the term “negative symptom” refers to a deficit in the ability of a subject to perform a normal function. Examples of negative symptoms, for example in a subject suffering from schizophrenia, include asociality, anhedonia, alogia, affective flattening, apathy, avolition, blunted affect, anergia, depression, low mood, and cognitive impairment. Negative symptoms, e.g., one or more of the foregoing negative symptoms, can also be experienced by subjects suffering from other diseases, for example subjects having a neurodegenerative disease, for example Parkinson's disease, Alzheimer's disease, or ALS. Common negative symptoms in such subjects include anhedonia, apathy, depression, and cognitive impairment.

As used herein, a “non-motor symptom” of Parkinson's disease refers to a symptom that is not related to movement. Non-motor symptoms include, for example, cognitive symptoms such as mood changes, depression, anxiety, panic attacks, tiredness, confusion, and slowed thinking, sensory symptoms such as numbness, restlessness, pain, chest discomfort, and anosmia, and autonomic symptoms such as hot/cold sensations, bladder problems, sweating, abdominal discomfort, constipation, sialorrhea, frequent urination and/or urgency, and erectile dysfunction.

As used herein, the terms “treat”, “treatment” and “treating” refer to the reduction or amelioration of the progression or severity of one or more symptoms (preferably, one or more discernible symptoms) of a disease, disorder, or condition resulting from the administration of a compound or composition of the disclosure (for avoidance of doubt, each AVPR1A agonist disclosed herein is considered a compound of the disclosure and pharmaceutical compositions comprising such AVPR1A agonists are considered compositions of the disclosure). In specific embodiments, the terms “treat”, “treatment” and “treating” refer to the amelioration of at least one measurable physical parameter of a disease, disorder, or condition. In other embodiments, the terms “treat”, “treatment” and “treating” refer to the slowing of the progression of one or more symptoms of a disease, disorder, or condition.

### Means for Agonizing AVPR1A

Agents compatible for use in methods of the invention generally comprise a means for agonizing AVPR1A. Such means include, e.g., an AVPR1A agonist as described herein.

### AVPR1A Agonists

AVPR1A agonists include peptides and non-peptide small molecules that bind to AVPR1A and trigger the activation of downstream signaling pathways. In some embodiments, AVPR1A agonists used in the methods and compositions of the disclosure reversibly bind to AVPR1A. While it is preferable that an AVPR1A agonist selectively binds to and activates AVPR1A, they do not need to be specific or selective for AVPR1A vs. other arginine-vasopressin receptors, such as AVPR1B and/or AVPR2, or oxytocin receptor (OTR). In some embodiments, an AVPR1A agonist of the disclosure selectively binds to and activates AVPR1A receptors vs. AVPR1B and AVPR2. In some embodiments, an AVPR1A agonist of the disclosure binds to and activates AVPR1A and optionally AVPR1B and/or AVPR2 and/or OTR. In some embodiments, the AVPR1A agonist is a selective AVPR1A agonist, for example an agonist that has greater selectivity for AVPR1A over AVPR2, for example selepressin. Selectivity of an agonist for different receptors can be assessed, for example, by comparing EC50 values.

An exemplary AVPR1A agonist is vasopressin, a natural ligand of AVPR1A. Accordingly, vasopressin can be used in the methods of the disclosure. Vasopressin analogs can also be used. Exemplary vasopressin analogs include 1-desamino-8-D-arginyl vasopressin (desmopressin or DDAVP), 8-D-homoarginine vasopressin, felypressin, ornipressin, selepressin, triglycyl lysine vasopressin (terlipressin), lypressin, F180 (Andres et al., 2002, Br. J. Pharmacol. 135(7):1828-36Cotte et al., 2003, Eur. J Biochem. 267, 4253-4263), and [deamino-Cys(1)]arginine vasopressin (dAVP). Analogs of the foregoing can also be used. In some embodiments, the AVPR1A agonist is felypressin.

An exemplary vasopressin analog has the general formula

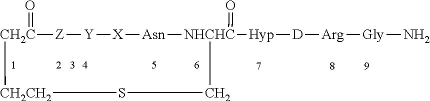

where X is (S)-2-amino-2-methyl-butanoic acid (CaMeAbu) or Valine (Val),
Y is Thienylalanine (Thi) or Methionine (Met),
Z is D-Phenylalanine (D-Phe) or D-Thienylalanine (Thi) or D-Tyrosine (D-Tyr)
Asn is Asparagine,
Hyp is 4-trans-Hydroxyproline,
D-Arg is D-Arginine, and
Gly is Glycine.

Vasopressin analogs characterized by the above formula are described in PCT publication no. WO 1995/000548 A1, which is incorporated by reference in its entirety.

Another exemplary vasopressin analog has the following general formula

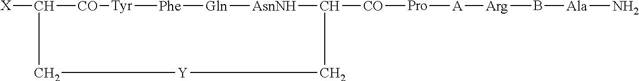

where X is selected from the group consisting of H and NH₂; Y is selected from the group consisting of —S—S—, —CH₂S— and —SCH₂—; and (i) A=L and B=D or (ii) A=D and B=L.

Vasopressin analogs characterized by the above formula are described in U.S. Pat. No. 4,285,858, which is incorporated by reference in its entirety.

Another exemplary vasopressin analog has the following general formula

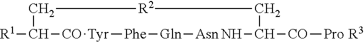

where:
R₁ is H, R₂ is CH₂—S and R₃ is D-Arg, or
R¹ NH₂, R² is S—S and R³ is selected from among D-Arg and L-Orn and all other chiral amino acids belonging to the L-series.

Vasopressin analogs characterized by the above formula are described in U.S. Pat. No. 4,482,486, which is incorporated by reference in its entirety.

Another exemplary vasopressin analog has the following general formula

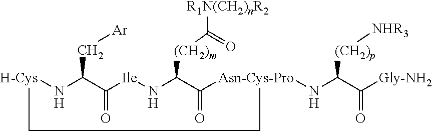

where:
Ar is an aryl group selected from aromatic carbocyclic ring systems, five- or six-membered heteroaromatic ring systems and bicyclic heteroaromatic ring systems;
m is selected from 1, 2 and 3;
n is selected from 0, 1, 2, 3 and 4;
p is selected from 2, 3 and 4;
R₁, R₂ and R₃ are independently selected from H, OH, alkyl, O-alkyl and OC(O)-alkyl; alkyl is selected from C₁₋₆ straight and C₄₋₈ branched chain alkyl and optionally has at least one hydroxyl substituent;
and when n=0, R₁ and R₂ optionally together form a nitrogen containing ring structure comprising from 2 to 5 carbon atoms;
with the proviso that when Ar is phenyl, m=2, n=0, R₁=R₂=H, R₃ is not H, and p is 3 or 4.

Vasopressin analogs characterized by the above formula are described in U.S. Pat. No. 8,222,202, which is incorporated by reference in its entirety.

Exemplary analogs of 8-D-homoarginine vasopressin are described in EP patent no. 0 412 508 B1, which is incorporated by reference in its entirety.

In some embodiments, the AVPR1A agonist is a vasopressin analog or analog of a vasopressin analog described in WO 1995/000548 A1, U.S. Pat. Nos. 4,285,858, 4,482,486, or U.S. Pat. No. 8,222,202, the contents of each which are incorporated herein by reference in their entireties.

In some embodiments, the AVPR1A agonist is not a peptide or peptide conjugate disclosed in WO 2016/140277 or WO 2018/047852, the contents of each which are incorporated herein by reference in their entireties.

### Pharmaceutical Compositions

In some aspects, the disclosure provides pharmaceutical compositions comprising an AVPR1A agonist as described herein. It should be understood that reference to a particular AVPR1A agonist encompasses the AVPR1A agonist and pharmaceutically acceptable salts thereof unless required otherwise by context. The pharmaceutical compositions are preferably formulated for oral administration. Pharmaceutical compositions can be prepared according to standard methods in the art (e.g., as described in Allen et al., eds., 2012, Remington: The Science and Practice of Pharmacy, 22^{nd} Edition, Pharmaceutical Press, London, UK).

In some aspects, the disclosure provides uses of an AVPR1A agonist, e.g., as described herein, in the manufacture of a medicament, for example, a medicament for treating a subject having or at risk of a disease or condition described herein.

### Methods of Treatment

AVPR1A agonists can be used to treat or prevent various diseases, disorders and conditions. This disclosure provides therapeutic uses for AVPR1A agonists, including for the treatment of diseases, disorders, and conditions amenable to treatment by vagus nerve stimulation and/or modulation of histamine and/or norepinephrine levels and/or modulation of the inflammatory cytokine TNF-α.

In some aspects, the disclosure provides a method for treating a subject suffering from or at risk of an inflammatory condition comprising administering an AVPR1A agonist to the subject in an amount effective to treat the subject. As would be understood by persons skilled in the art, various conditions have inflammatory aspects and can be considered inflammatory conditions, for example, inflammatory bowel disease, rheumatoid arthritis, and schizophrenia.

In some aspects, the disclosure provides a method for treating a subject suffering from or at risk of a disease or disorder of the nervous system (e.g., the central nervous system) comprising administering an AVPR1A agonist to the subject in an amount effective to treat the subject.

In some aspects, the disclosure provides a method for treating a subject suffering from or at risk of schizophrenia or psychosis comprising administering an AVPR1A agonist to the subject in an amount effective to treat the subject. In some embodiments, the subject is suffering from schizophrenia. In some embodiments, the subject exhibits psychotic behavior.

Schizophrenia is characterized by positive symptoms (e.g., delusions and hallucinations), negative symptoms, abnormalities in mood, and cognition deficits, and often leads to severe functional impairment (Yasui-Furukori, 2012, Drug Des Devel Therapy 6:107-115). The treating can comprise, for example, improving or slowing progression of one or more negative symptoms of schizophrenia or psychosis. Negative symptoms include asociality, anhedonia, alogia, affective flattening, apathy, avolition, blunted affect, anergia, apathy, depression, low mood, and cognitive impairment. The cognitive impairment can be, for example, a deficit in verbal working memory, spatial working memory, verbal fluency, verbal learning, or a combination thereof. Negative symptoms, and changes in such symptoms, can be assessed by various scales and scores, for example, Positive and Negative Syndrome Scale (PANSS) (Kay et al., 1987, Schizophr Bull. 13(2):261-76), Scale for the Assessment of Negative Symptoms (SANS) (Andreasen, 1982, Arch Gen Psychiatry 39(7):784-8), Personal and Social Performance (PSP) scale (Morosini et al., 2000, Acta Psychiatr Scand. 101(4):323-9), Clinical Global Impression Improvement (CGI-I) scale (Busner & Targum, 2007, Psychiatry (Edgmont). 4(7):28-37), and Clinical Global Impression Severity (CGI-S) scale (Busner & Targum, 2007, Psychiatry (Edgmont). 4(7):28-37). Treating a subject with an AVPR1A agonist can comprise improving or slowing worsening of one or more negative symptoms as measured by one or more of these scales or scores, or a portion thereof.

In another aspect, the disclosure provides a method for treating a subject suffering from a neurodegenerative disease such as Parkinson's disease, Alzheimer's disease, or amyotrophic lateral sclerosis (ALS), comprising administering to the subject an AVPR1A agonist in an amount effective to treat the subject. The treating can comprise improving or slowing progression of one or more negative symptoms of the neurodegenerative disease, for example asociality, anhedonia, alogia, affective flattening, apathy, avolition, blunted affect, anergia, depression, low mood, and cognitive impairment. Negative symptoms, and changes in such symptoms, can be assessed by various scales and scores, for example those described in the preceding paragraph.

In some aspects, the disclosure provides a method for treating a subject suffering from Parkinson's disease comprising administering to the subject an AVPR1A agonist in an amount effective to treat the subject. The treating can comprise improving or slowing progression of one or more non-motor symptoms of Parkinson's disease. Non-motor symptoms of Parkinson's disease include sensory symptoms (e.g., numbness, restlessness, pain, chest discomfort, anosmia), cognitive symptoms (e.g., mood changes, depression, anxiety, panic attacks, tiredness, confusion, slowed thinking), and autonomic symptoms (e.g., hot/cold sensations, bladder problems, sweating, abdominal discomfort, constipation, sialorrhea (drooling or excessive salivation), frequent urination and/or urgency, erectile dysfunction) (see, e.g., Barone et al., 2009, Mov Disord. 24(11):1641-9). Non-motor symptoms, and changes in such symptoms, can be assessed by various scales and scores, for example, the Non-motor Symptoms Scale (NMSS) (Chaudhuri et al., 2007, Movement Disorders 22(13):1901-11), Movement Disorder Society Unified Parkinson's Disease Rating Scale (MDS-UPDRS) non-motor symptoms score (Goetz et al., International Parkinson and Movement Disorder Society, Aug. 13, 2019 update; www.movementdisorders.org/MDS-Files1/PDFs/MDS-UPDRS_English_FINAL.pdf)), Fatigue Severity Scale (FSS) score (Krupp et al., 1989, Arch Neurol. 46(10):1121-3), Beck's Depression Inventory II (BDI-II) score (Beck, Steer, & Brown, 1996), Beck Anxiety Inventory (BAI) score (Beck et al., 1988, J Consult Clin Phychol. 56(6):893-7), Montreal Cognitive Assessment (MoCA) score (Nasreddine et al., 2005, J Am Geriatr Soc. 53(4):695-9), scores on Cogstate test(s) (cogstate.com), Clinical Global Impression Improvement (CGI-I) overall score or non-motor symptoms score (Busner & Targum, 2007, Psychiatry (Edgmont). 4(7):28-37), Clinical Global Impression Severity (CGI-S) overall or non-motor symptoms score (Busner & Targum, 2007, Psychiatry (Edgmont). 4(7):28-37), and Parkinson's Disease Questionnaire-39 (PDQ-39) (Jenkinson et al., 1997, Age Ageing. 26(5):353-7). Cogstate tests include the following computerized cognitive assessments: Behavioral Pattern Separation Object test; Continuous Paired Associate Learning Test; Face Name Associative Memory Exam; Groton Maze Learning Test; Identification Test; International Daily Symbol Substitution Tests (medicines or symbols); International Shopping List Test; One Back Test; One Card Learning Test; Sustained Attention Test; Sustained Attention to Response Test; and Two Back Test.

In some aspects, the disclosure provides a method for improving or slowing progression of a cognitive deficit in a subject or improving cognition is a subject not having a cognitive deficit comprising administering an AVPR1A agonist to the subject in an amount effective to improve cognition or slow progression of a cognitive deficit.

In another aspect, the disclosure provides a method for treating a subject suffering from gastrointestinal disease or condition comprising administering an AVPR1A agonist to the subject in an amount effective to treat the subject.

In another aspect, the disclosure provides a method for treating a subject suffering from irritable bowel syndrome comprising administering an AVPR1A agonist to the subject in an amount effective to treat the subject.

In another aspect, the disclosure provides a method for treating a subject suffering from inflammatory bowel disease comprising administering an AVPR1A agonist to the subject in an amount effective to treat the subject.

In another aspect, the disclosure provides a method for treating a subject suffering from ulcerative colitis comprising administering an AVPR1A agonist to the subject in an amount effective to treat the subject.

In another aspect, the disclosure provides a method for treating a subject suffering from Crohn's disease comprising administering an AVPR1A agonist to the subject in an amount effective to treat the subject.

In another aspect, the disclosure provides a method for treating a subject suffering from constipation comprising administering an AVPR1A agonist to the subject in an amount effective to treat the subject.

In another aspect, the disclosure provides a method for treating a subject suffering from pain comprising administering an AVPR1A agonist to the subject in an amount effective to treat the subject.

In another aspect, the disclosure provides a method for treating a subject suffering from visceral pain comprising administering an AVPR1A agonist to the subject in an amount effective to treat the subject.

In another aspect, the disclosure provides a method for treating a subject suffering from rheumatoid arthritis comprising administering an AVPR1A agonist to the subject in an amount effective to treat the subject.

In another aspect, the disclosure provides a method for treating a subject suffering from migraine comprising administering an AVPR1A agonist to the subject in an amount effective to treat the subject.

In another aspect, the disclosure provides a method for treating a subject suffering from headache comprising administering an AVPR1A agonist to the subject in an amount effective to treat the subject.

In another aspect, the disclosure provides a method for treating a subject suffering from substance abuse (e.g., substance use disorder such as drug use disorder, polysubstance use disorder, alcohol use disorder, nicotine use disorder, or tobacco use disorder) comprising administering an AVPR1A agonist to the subject in an amount effective to treat the subject. In some embodiments, the subject has drug use disorder. In some embodiments, the subject has polysubstance use disorder. In some embodiments, the subject has alcohol use disorder. In some embodiments, the subject has nicotine use disorder. In some embodiments, the subject has tobacco use disorder.

In another aspect, the disclosure provides a method for treating a subject suffering from drug addiction comprising administering an AVPR1A agonist to the subject in an amount effective to treat the subject. Examples of drug addiction include opioid addiction, for example addiction to morphine, heroin, oxycodone, or fentanyl; cocaine addiction; and benzodiazepine addiction such as addiction to diazepam, alprazolam, or clonazepam.

In another aspect, the disclosure provides a method for treating a subject suffering from a seizure disorder comprising administering an AVPR1A agonist to the subject in an amount effective to treat the subject. In some embodiments, the seizure disorder is epilepsy. In some embodiments, the seizure disorder is an orphan epilepsy (see, e.g., Perucca et al., 2020, Lancet 19:544-556).

In another aspect, the disclosure provides a method for treating a subject suffering from major depressive disorder comprising administering an AVPR1A agonist to the subject in an amount effective to treat the subject.

In another aspect, the disclosure provides a method for treating a subject suffering from atypical depression comprising administering an AVPR1A agonist to the subject in an amount effective to treat the subject.

In another aspect, the disclosure provides a method for treating a subject suffering from a major depressive episode (MDE) (e.g., atypical MDE) comprising administering an AVPR1A agonist to the subject in an amount effective to treat the subject.

In another aspect, the disclosure provides a method for treating a subject suffering from depression in the presence of a neurodegenerative disease (e.g., Parkinson's disease, Alzheimer's disease, or ALS) comprising administering an AVPR1A agonist to the subject in an amount effective to treat the subject. Depression in subjects having a neurodegenerative disease is typically resistant to traditional anti-depressant therapy (Hussain et al., 2020, Cureus 12(11): e11613),

In another aspect, the disclosure provides a method for treating a subject suffering from treatment-resistant depression comprising administering an AVPR1A agonist to the subject in an amount effective to treat the subject.

In another aspect, the disclosure provides a method for treating a subject suffering from cognitive impairment comprising administering an AVPR1A agonist to the subject in an amount effective to treat the subject.

In another aspect, the disclosure provides a method for treating a subject suffering from Alzheimer's disease comprising administering an AVPR1A agonist to the subject in an amount effective to treat the subject.

In another aspect, the disclosure provides a method for treating a subject suffering from COVID-19 related cognitive impairment and/or depression comprising administering an AVPR1A agonist to the subject in an amount effective to treat the subject.

In another aspect, the disclosure provides a method for treating a subject suffering from ADHD comprising administering an AVPR1A agonist to the subject in an amount effective to treat the subject.

In another aspect, the disclosure provides a method for treating a subject suffering from an autism spectrum disorder comprising administering an AVPR1A agonist to the subject in an amount effective to treat the subject.

In another aspect, the disclosure provides a method for treating a subject suffering from a pervasive developmental disorder, such as Asperger syndrome or Rett syndrome, comprising administering an AVPR1A agonist to the subject in an amount effective to treat the subject.

In another aspect, the disclosure provides a method for treating a subject suffering from pervasive developmental disorders not otherwise specified comprising administering an AVPR1A agonist to the subject in an amount effective to treat the subject.

In another aspect, the disclosure provides a method for treating a subject suffering from multiple sclerosis comprising administering an AVPR1A agonist to the subject in an amount effective to treat the subject.

In another aspect, the disclosure provides a method for treating a subject suffering from PTSD comprising administering an AVPR1A agonist to the subject in an amount effective to treat the subject.

In another aspect, the disclosure provides a method for treating a subject suffering from a sleep disorder comprising administering an AVPR1A agonist to the subject in an amount effective to treat the subject.

In another aspect, the disclosure provides a method for treating a subject suffering from insomnia comprising administering an AVPR1A agonist to the subject in an amount effective to treat the subject.

In another aspect, the disclosure provides a method for treating a subject suffering from daytime fatigue comprising administering an AVPR1A agonist to the subject in an amount effective to treat the subject.

In another aspect, the disclosure provides a method for treating a subject suffering from REM sleep behavior disorder comprising administering an AVPR1A agonist to the subject in an amount effective to treat the subject.

In another aspect, the disclosure provides a method for improving sleep in a subject comprising administering an AVPR1A agonist to the subject in an amount effective to improve the subject's sleep.

In another aspect, the disclosure provides a method for treating a subject suffering from or at risk of a mental disorder comprising administering an AVPR1A agonist to the subject in an amount effective to treat the subject. The mental disorder is in some embodiments a mood disorder, an anxiety disorder, or a disorder of diminished motivation. In some embodiments, the mental disorder is depression, bipolar disorder, or adjustment disorder. In some embodiments, the mental disorder is an anxiety disorder. In some embodiments, the mental disorder is a disorder of diminished motivation, e.g., apathy, abulia, or akinetic mutism.

In another aspect, the disclosure provides a method for improving cognitive function of a subject comprising administering an AVPR1A agonist to the subject in an amount effective to improve cognitive function. The subject may be a healthy subject, e.g., one that does not suffer from a condition described herein.

In another aspect, the disclosure provides a method for improving mood of a subject comprising administering an AVPR1A agonist to the subject in an amount effective to improve mood. The subject may be a healthy subject, e.g., one that does not suffer from a condition described herein.

In another aspect, the disclosure provides a method for improving or slowing worsening of an overall Patient Health Questionnaire-9 (PHQ-9) score of a subject comprising administering an AVPR1A agonist to the subject in an amount effective to improve or slow worsening of the subject's overall PHQ-9 score. The subject may be a healthy subject, e.g., one that does not suffer from a condition described herein.

In another aspect, the disclosure provides a method for improving or slowing worsening of a subject's score on one or more of questions of PHQ-9, such as PHQ-9 questions 1, 2, 3, or 6, comprising administering an AVPR1A agonist to the subject in an amount effective to improve or slow worsening of the subject's score on the PHQ-9 question(s).

In another aspect, the disclosure provides a method for improving or slowing worsening of one or more of the CogState Groton Maze Learning Test (GMLT), the CogState Identification Test (IDN), or the CogState One Card Learning Test (OCL) results of a subject, comprising administering an AVPR1A agonist to the subject in an amount effective to improve or slow worsening of the subject's result(s) on the GMLT, IDN, and/or OCL. The subject may be a healthy subject, e.g., one that does not suffer from a condition described herein.

In another aspect, the disclosure provides a method for improving or slowing worsening of at least one of the alpha band power or the gamma band power of an electroencephalogram (EEG) of a subject. The subject may be a healthy subject, e.g., one that does not suffer from a condition described herein.

In some embodiments of the methods described herein, the subject has an elevated TNF-α level, e.g., relative to a reference range obtained from healthy subjects. For example, in some embodiments, a subject having an elevated TNF-α level has a serum TNF-α level greater than 5.6 pg/ml, for example as measured by a sandwich immunoassay (e.g., the Eurofins Viracor TNF-α serum test; www.eurofins-viracor.com/clinical/test-menu/1220-tnf-alpha-tnf-a-serum.

The subjects of the methods described herein are preferably mammals, e.g., humans or domestic pets (e.g., cat, dog). Subjects can be of any age, but are preferably adults (e.g., a human subject who is 18 years old or more, 25 years old or more, 35 years old or more, 45 years old or more, 55 years old or more, etc.). In some embodiments, the subject is elderly (e.g., a human subject who is 65 years old or more, 70 years old or more, 75 years old or more, or 80 years old or more).

AVPR1A agonists and pharmaceutical compositions containing them can be administered orally, topically, rectally, or parenterally, with oral administration being preferred. The method of administration can vary depending on the condition and age of the subject.

Appropriate daily dosages of AVPR1A agonists can range from 0.005 mg/kg to 500 mg/kg of body weight per day (e.g., 0.005 mg/kg to 100 mg/kg, 0.005 mg/kg to 30 mg/kg, 0.005 mg/kg to 1 mg/kg, 0.01 mg/kg to 30 mg/kg, 0.01 mg/kg to 3 mg/kg, 0.01 mg/kg to 1 mg/kg, 0.02 mg/kg to 5 mg/kg, 0.02 mg/kg to 2 mg/kg, 0.02 mg/kg to 1 mg/kg). Alternatively, the compounds can be administered at a fixed dose ranging from 0.1 mg to 50 g per day (e.g., 0.1 mg to 10 g, 0.1 mg to 3 g, 0.1 mg to 100 mg, 0.1 mg to 1 mg, 0.3 mg to 3 g, 0.3 mg to 100 mg, 20 mg to 2 g, 20 mg to 1 g, 0.5 g to 2 g, 0.5 g to 1 g, or 1 g to 2 g). For administration of a pharmaceutical composition, an amount of the pharmaceutical composition can be administered that contains an amount of the AVPR1A agonist that is within one of the foregoing ranges. A daily dose can be administered as a single dose or multiple doses.

An AVPR1A agonist can be administered as monotherapy, or in combination with one or additional therapeutic agents, e.g., one or more agents which are not AVPR1A agonists.

### EXAMPLES

### Example 1: LSSTQAQQSY (SEQ ID NO:1) Target Characterization

Several screens were performed to characterize the molecular target of LSSTQAQQSY (SEQ ID NO: 1). Initial screens including screening of Trp channels (TRPA1, TRPV1), nutrient and metabolite sensing GPCRs (FFAR1, FFAR2, FFAR3, FFAR4, GPR119, CaS, GPR142, GPBAR1 and LPA5) and taste receptors; a full genome-wide binding-based screen of all cell surface-expressed receptors (~6K receptors); and a large screen of 168 GPCRs for agonist or antagonist activity. The initial screens were negative or provided non-reproducible hits.

Subsequently, a more universal and more sensitive GPCR screening strategy was employed. In the screen, three possible targets were identified: GNRHR, AVPR1A, and AVPR2. Studies were then performed to identify and confirm the target.

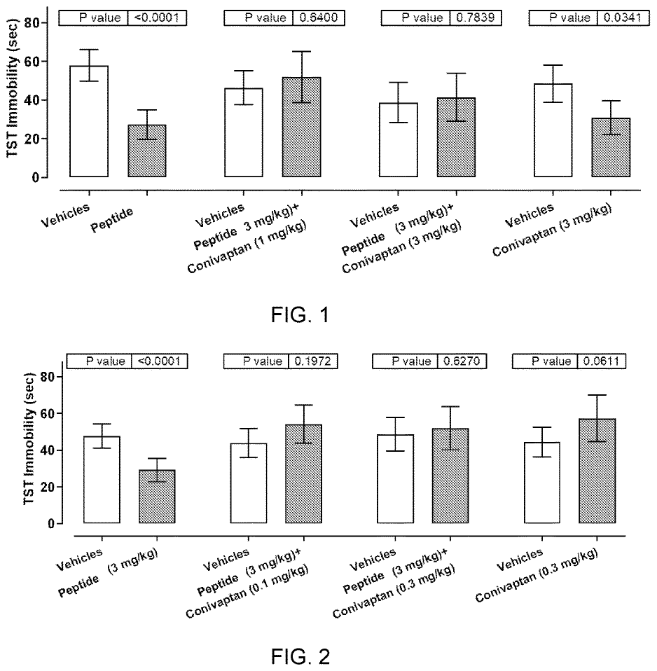

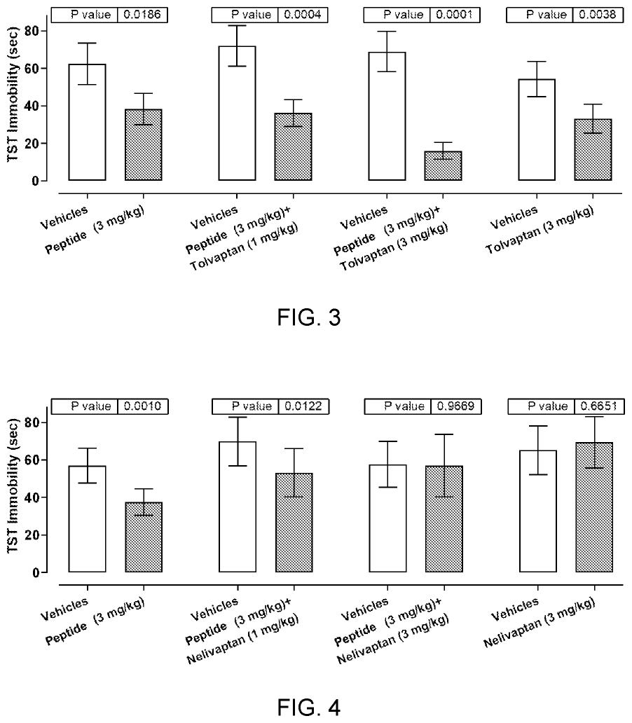

It was found that the effect of LSSTQAQQSY (SEQ ID NO: 1) was blocked by the AVPR1A/AVPR2 inhibitor conivaptan in a tail suspension test (TST) (see, WO 2016/140277), suggesting that the primary target of LSSTQAQQSY (SEQ ID NO: 1) is either AVPR1A or AVPR2 (FIGS. 1-2). It was found that the effect of LSSTQAQQSY (SEQ ID NO: 1) was not blocked by the AVPR2 inhibitor tolvaptan (FIG. 3), suggesting that the primary target of the peptide is not AVPR2. It was also found that the effect of LSSTQAQQSY (SEQ ID NO: 1) was blocked by the AVPR1A/B inhibitor nelivaptan (FIG. 4), indicating that the primary target of the peptide is AVPR1A.

The results of the studies are summarized in Table 1.

TABLE 1

| | | | Peptide | Peptide | |
|---|---|---|---|---|---|
| | | | activity | activity | |
| | | Peptide activity | blocked by | blocked by | |
| | Activated by | blocked by | tolvaptan | nelivaptan | |
| | peptide in in | conivaptan | (AVPR2 | (AVPR1A/1B | |
| Candidate | vitro | (AVPR1A/AVPR2 | inhibitor) in | inhibitor) in | Likely |
| receptor | screens? | inhibitor) in vivo? | vivo? | vivo? | target |
| AVPR1A | Yes | Yes | NA | Yes | Yes |
| AVPR1B | No | NA | NA | Yes | No |
| AVPR2 | Yes | Yes | No | NA | No |

### Example 2: LSSTQAQQSY (SEQ ID NO:1) Brain Microdialysis Study

A study was performed to determine the CNS neurotransmitter responses elicited by oral dosing with the peptide LSSTQAQQSY (SEQ ID NO: 1).

Male C57Bl/6 mice were fitted with microdialysis probes in the prefrontal cortex (which modulates sensory processing, memory, and emotions) or striatum (required for behavioral reinforcement by natural rewards). Animals were orally administered a single 3 mg/kg dose of peptide or vehicle. Brain dialysate samples were collected before administration and every 30 minutes for four hours after. Dopamine, norepinephrine, serotonin, histamine, glutamate, GABA, and glycine concentrations in dialysate samples were determined by HPLC-MS.

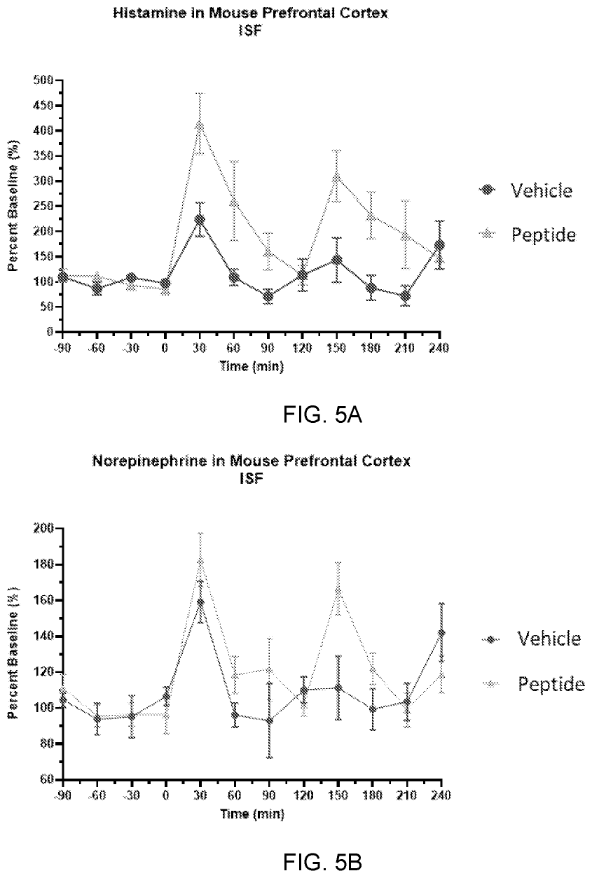

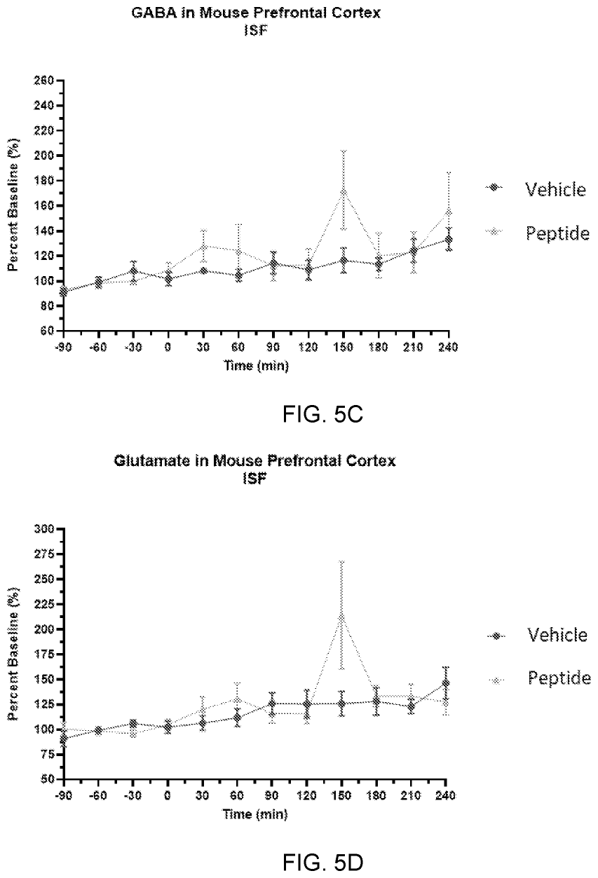

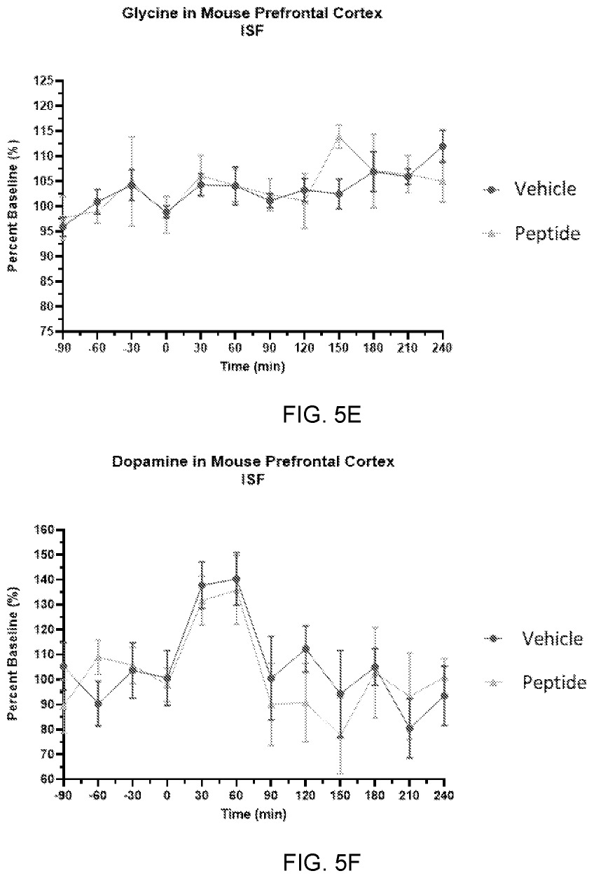

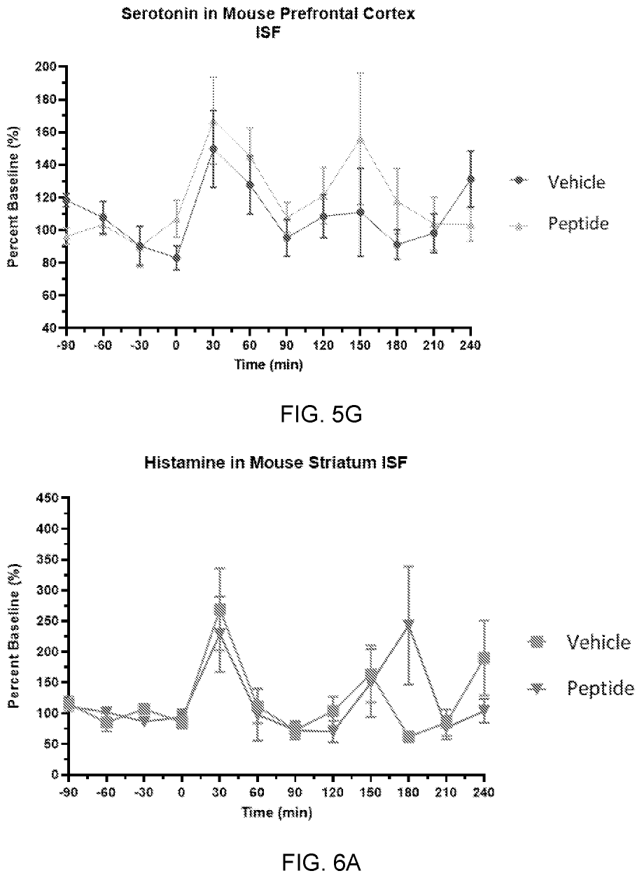

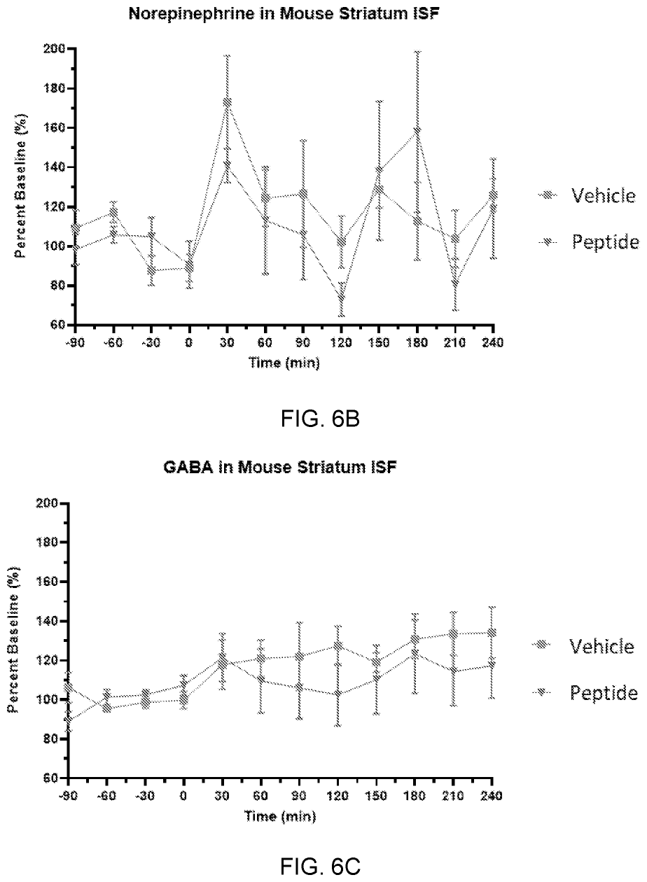

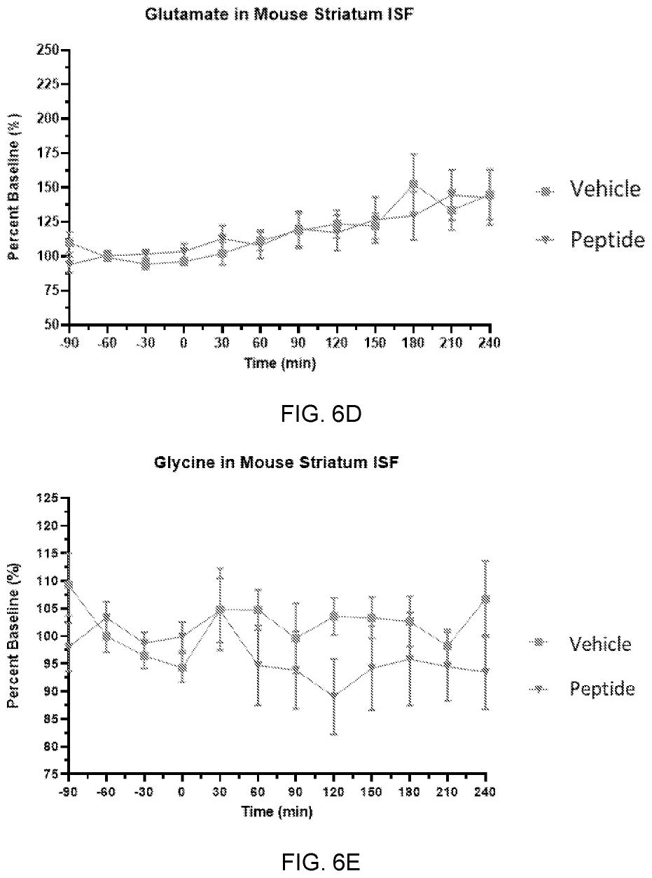

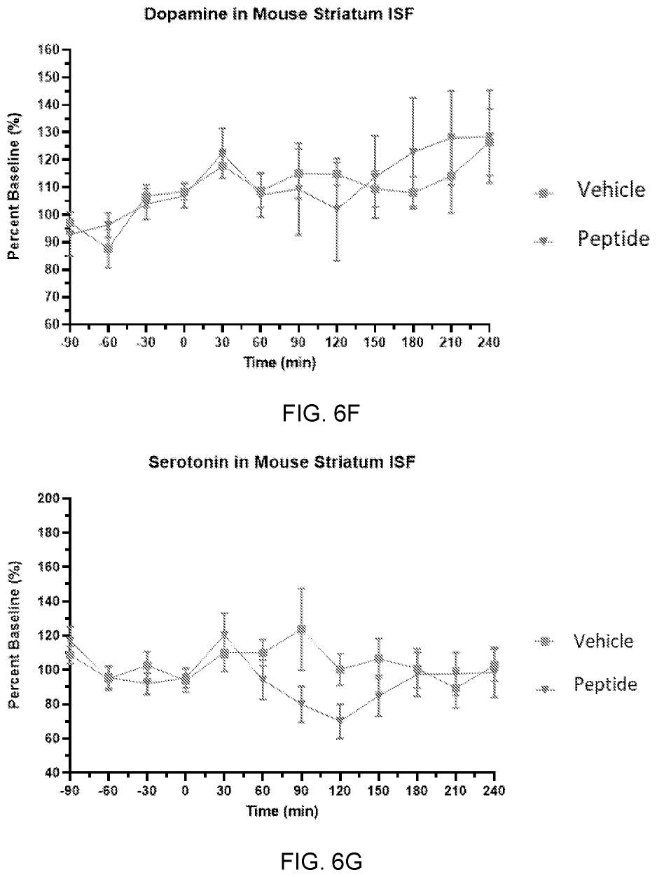

Results are shown in FIGS. 5A-5G and FIGS. 6A-6G.

Statistically significant changes in histamine and norepinephrine in the prefrontal cortex were observed. No significant changes were observed with other neurotransmitters in the prefrontal cortex and all evaluated neurotransmitters in striatum.

### Example 3: Inflammation Suppression by LSSTQAQQSY (SEQ ID NO:1)

Vagus nerve stimulation has been reported to suppress lipopolysaccharide (LPS)-induced increases in serum levels of the inflammatory cytokine TNF-α (see, e.g., Tarnawski et al., 2018, Front. Immunol. 9:2648; Komegae et al., 2018, Brain Behav Immun. 73:441-449). As the peptide LSSTQAQQSY (SEQ ID NO: 1) is believed to act via the vagus nerve, the ability of the peptide to suppress TNF-α following LPS injection was assessed.

Mice were administered LPS at a dose of 10 μg/kg, i.p. The peptide LSSTQAQQSY (SEQ ID NO: 1) was administered twice to the mice at 0.3 mg/kg, 3 mg/kg, or 10 mg/kg, p.o., 2.5 and 3.5 hours the after LPS administration. Blood was collected 4 hours after the LPS administration. Plasma levels of TNF-α were subsequently measured by ELISA.

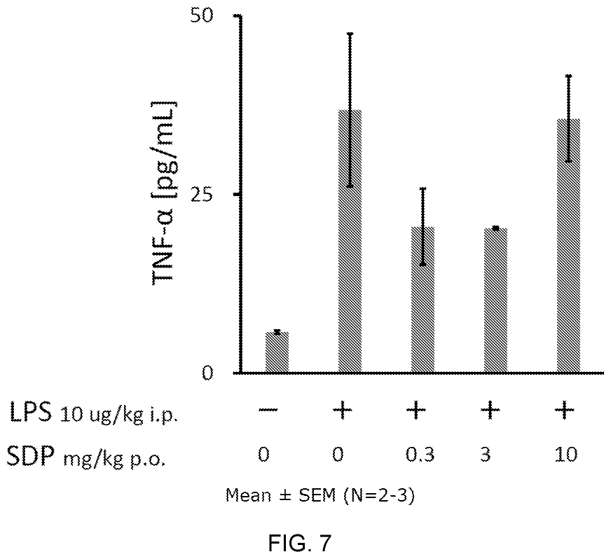

Results are shown in FIG. 7. A trend of TNF-α suppression was observed in animals administered the peptide following in vivo challenge with LPS, indicating an anti-inflammatory effect of the peptide.

### Example 4: Mood and Cognition Improvement in Healthy Subjects in Phase 1a/1b Study

A Phase 1a/1b, randomized, placebo-controlled, crossover study with the peptide LSSTQAQQSY (SEQ ID NO: 1) in healthy subjects was performed. The study population included men and women in good general health.

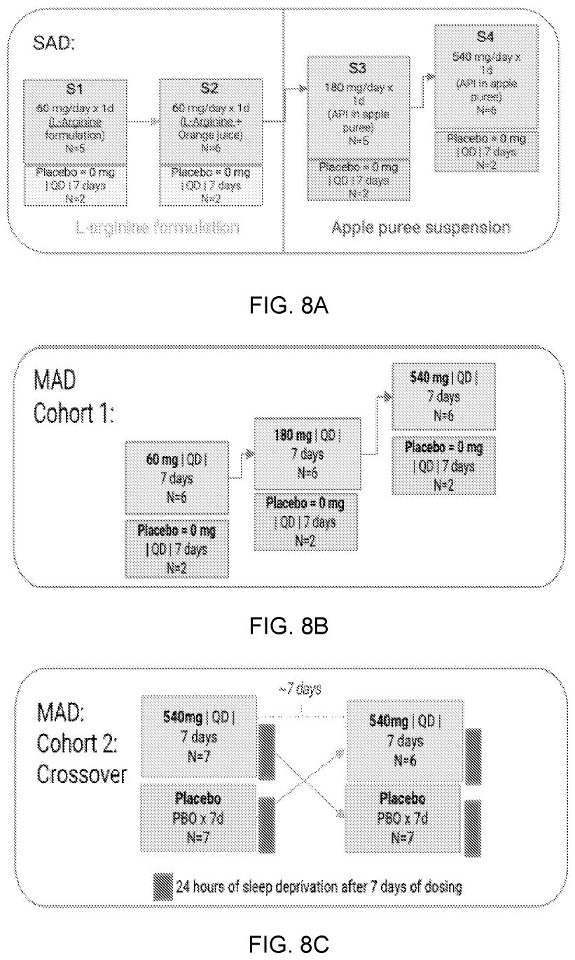

A single ascending dose (SAD) group included four cohorts receiving 60 mg/day (cohorts SAD1 and SAD2), 180 mg/day (cohort SAD3), or 540 mg/day (cohort SAD4) of the peptide or placebo. (See FIG. 8A.) A first multiple ascending dose (MAD) cohort (MAD Cohort 1) included three groups, each receiving 60 mg/day (MAD1), 180 mg/day (MAD2), or 540 mg/day MAD3) of the peptide or placebo for seven days. All doses were administered orally. (See FIG. 8B.) A second MAD cohort (MAD Cohort 1) employed a crossover study design with one group (n=7) of participants starting with 540 mg of peptide once a day for 7 days, followed by a 24-hour period of sleep deprivation. Seven days after the end of dosing, this group was then switched to receiving placebo once a day for seven days, followed by another 24-hour period of sleep deprivation. then crossing to 540 mg of peptide once a day for 7 days. In another group (n=7, with one participant withdrawing after initiation for personal reasons), the inverse dose regimen was used, with participants starting with placebo and the crossing over to 540 mg of peptide once a day. (See FIG. 8C.) Subjects were tested after the 24 hours of sleep deprivation.

### Results

### Safety

A few members of each cohort experienced mild adverse effects, generally gastrointestinal upset or throat irritation. Vital signs, electrocardiography (ECG) data, and physical examinations were normal for all participants throughout the study, except that in MAD Cohort 1, two subjects missed assessments due to positive COVID test and in MAD Cohort 2, one participant left the study for personal reasons.

Patient Questionnaires—Results from MAD Cohort 1

Patient Health Questionnaire-9 (PHQ-9), a standard questionnaire for assessing depression severity, was administered to the subjects in groups MAD1-MAD3 about four hours after each of the placebo and peptide administration periods. Each of the nine questions asks the subject how often in the previous two weeks he or she has/had the following:

| Question # | |
|---|---|
| 1 | Little interest or pleasure in doing things? |
| 2 | Feeling down, depressed, or hopeless? |
| 3 | Trouble falling or staying asleep, or sleeping too much? |
| 4 | Feeling tired or having little energy? |
| 5 | Poor appetite or overeating? |
| 6 | Feeling bad about yourself - or that you are a failure or have let yourself or your |
| | family down? |
| 7 | Trouble concentrating on things, such as reading the newspaper or watching |
| | television? |
| 8 | Moving or speaking so slowly that other people could have noticed? Or the |
| | opposite - being so fidgety or restless that you have been moving around a lot more |
| | than usual? |
| 9 | Thoughts that you would be better off dead or of hurting yourself in some way? |

Each question is answered on a 0 to +3 scale, with 0 for not at all, +1 for several days, +2 for more than half the days, and +3 for nearly every day.

Whether the peptide led to improvement over placebo in each PHQ-9 question was assessed.

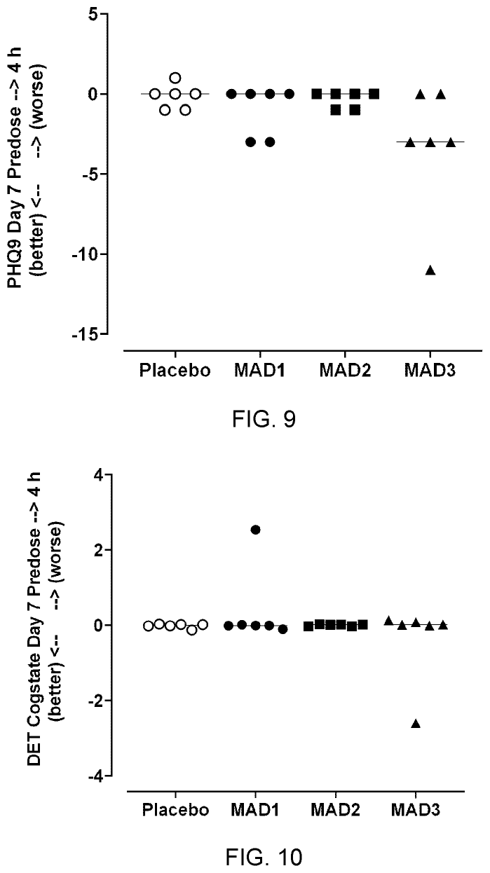

Preliminary data indicate that administration of the peptide led to improvement in measures of mood, depression, sleep, awareness, and/or alertness in group MAD3 (improvement in answers to PHQ-9 questions 1-3 and 6, with overall PHQ-9 score summarized in FIG. 9).

### CogState

The subjects in groups MAD1-MAD3 also underwent cognitive assessments (CogState Ltd., Melbourne, Vic., Australia): 1) Detection Test (DET), an assessment of psychomotor function; 2) Groton Maze Learning Test (GMLT), an assessment of executive function; 3) Identification Test (IDN), an assessment of attention; and 4) One Card Learning Test (OCL), an assessment of visual learning. The cognitive assessments were performed at essentially the same times as administration of the PHQ-9 questionnaire to the subjects. Whether the peptide led to improvement over placebo in the four cognitive assessments was qualitatively determined.

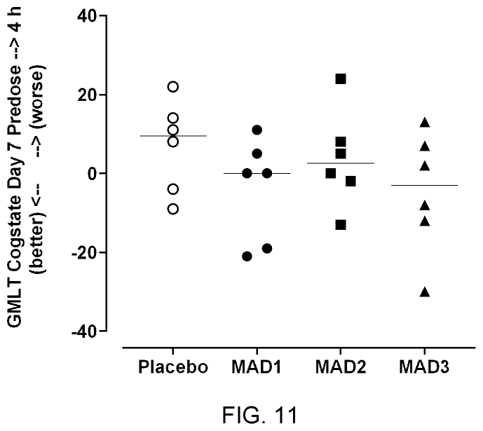

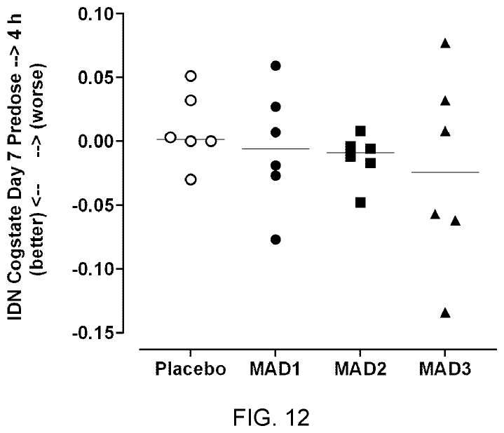

Preliminary data indicate that the peptide did not have a significant impact on psychomotor function in healthy subjects (FIG. 10). Preliminary data further indicate a positive trend in GMLT (FIG. 11), IDN (FIG. 12), and OCL (not shown) after seven days of treatment.

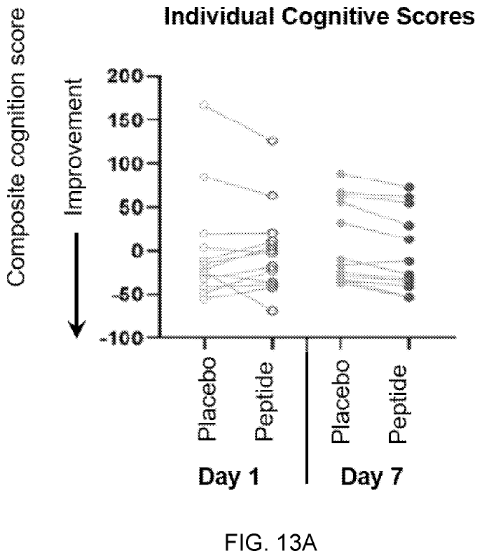

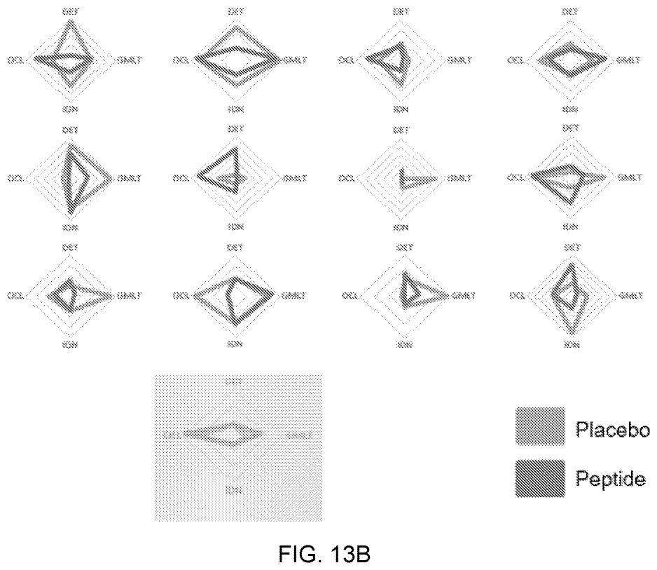

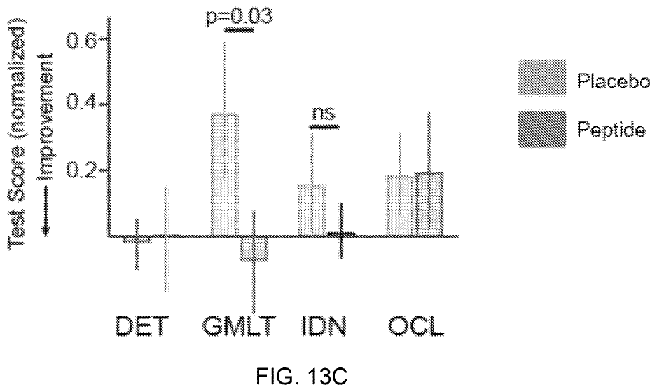

FIG. 13A shows composite cognition scores in the placebo group and in the peptide-treated groups in MAD Cohort 2 (the crossover study) at day 1 (pre-dosing) and day 7 (at four hours post-dosing). On day 1, no statistically significant difference was observed between the cognition scores in the placebo and the peptide-treated groups. (Five out of 13 peptide-treated subjects had a better score than placebos.) On day 7, twelve out of thirteen peptide-treated subjects had an improved composition cognition score (p<0.01).

FIG. 13B shows radar plots for the 13 subjects in MAD Cohort 2 for the four Cogstate tests: detection test (DET), Groton Maze Learning (GMLT), Identification test (IDN), and One Card Learning test (OCL). FIG. 1 shows test scores in the four tests in placebos (left-most bars within each pair of bars for each test) and peptide-treated subjects (right-most bars within each pair of bars for each test). Smaller scores indicated better cognition. Twelve of the thirteen subjects improved compared to the placebo on at least one of the four tests. Test improvement was most consistent in the Groton Maze Learning test (n=9/13; p<0.03)

Together, these results confirmed that seven days of dosing with the peptide improved the cognition scores in healthy volunteers.

In conclusion, data from the study indicate that treatment with the peptide improved executive function, attention, and visual learning in the subjects.

### Alpha and Gamma Electroencephalography (EEG) Bands—MAD Cohort 1

Quantitative EEG (qEEG) metrics can be used as a biomarker of Alzheimer's disease. In Alzheimer's, Parkinson's, and mild cognitive impairment (MCI), EEG changes include an increase in theta power compared to that in age-matched healthy controls and a decrease in alpha power compared to that in age-matched healthy controls. (See, e.g., Meghdadi A H, et al., “Resting state EEG biomarkers of cognitive decline associated with Alzheimer's disease and mild cognitive impairment.” PloS One. 2021 Feb. 5; 16(2).)

Various bands observed in EEG are associated with various states of mental activity, generally as summarized on the following table:

| Band name | Frequency Range (Hz) | Mental Activity |
|---|---|---|
| Delta | 1.5-6 | Sleep, dreaming |
| Theta | 6-8.5 | Drowsiness |
| Alpha | 8.5-12.5 | Relaxed, aware |
| Beta | 12.5-30 | Alert, attentive |
| Gamma | 30-40 | Focus, concentration |

Across the MAD groups, quantitative EEG (qEEG) was recorded for some subjects shortly before administering the final dose (day 7) and about 1 hr, about 2 hr, about 4 hr, about 8 hr, and about 12 hr after administering the final dose. The power in each band recorded shortly before administering the first dose (day 10 provided a baseline (100%) against which the post-dose EEG band power was normalized).

Preliminary data from the MAD1 group, in which each dose contained 60 mg of the peptide, indicated pronounced increases in power in the alpha band, from about 80% of baseline before administering the final dose to about 150% of baseline only 1 hr after the final dose. Alpha band power varied from about 120% to about 200% until about 12 hr after the final dose. Gamma band power similarly increased from about 100-110% of baseline before administering the final dose to about 140% of baseline 1 hr after the final dose, and remained above about 125% of baseline for 8 hr.

Preliminary data from the MAD2 group, in which each dose contained 180 mg of the peptide, also indicated pronounced increases in power in the alpha and gamma bands. Alpha band power increased from about 110% of baseline before administering the final dose to about 150% of baseline 2 hr after the final dose, and remained above 110% of baseline until about 12 hr after the final dose. Gamma band power increased from about 100-110% of baseline before administering the final dose to about 125% of baseline 1 hr after the final dose, and was above about 150% of baseline after 12 hr.

Preliminary data from the MAD3 group, in which each dose contained 540 mg of the peptide, indicated decreases in alpha band power from about 100% of baseline before administering the final dose to about 70-80% of baseline from 1 hr to 12 hr after administering the final dose.

In summary, preliminary data from the study shows that treatment with the peptide at doses of 60 mg/day or 180 mg/day increased the power of alpha and gamma EEG bands, indicative of states of relaxed awareness and focused concentration.

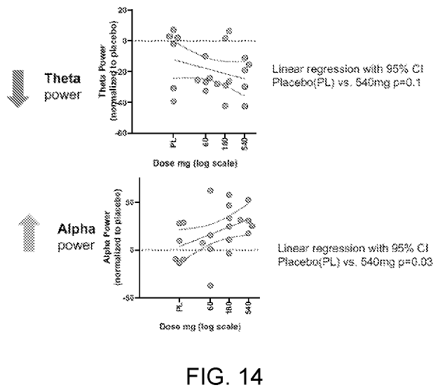

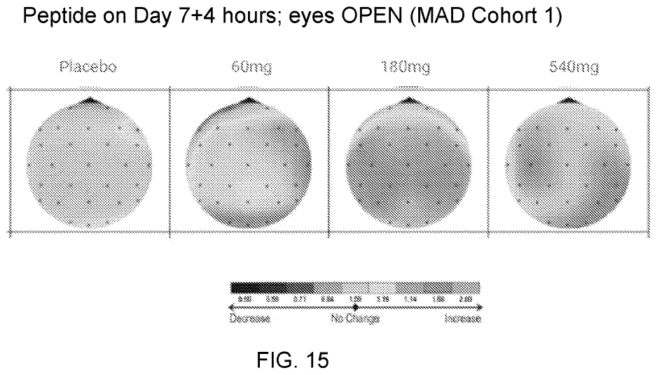

FIG. 14 depicts the theta power and alpha power across the MAD groups having different dose levels. FIG. 15 depicts the Alpha power levels in eyes open and closed states across the MAD groups having different dose levels.

### Sleep Quality Assessments

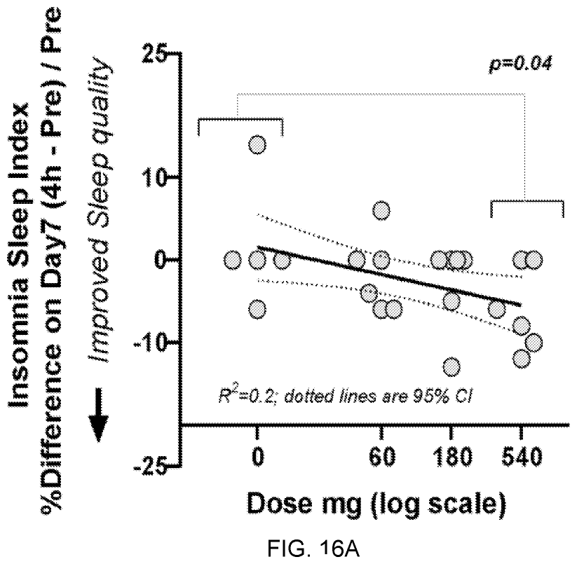

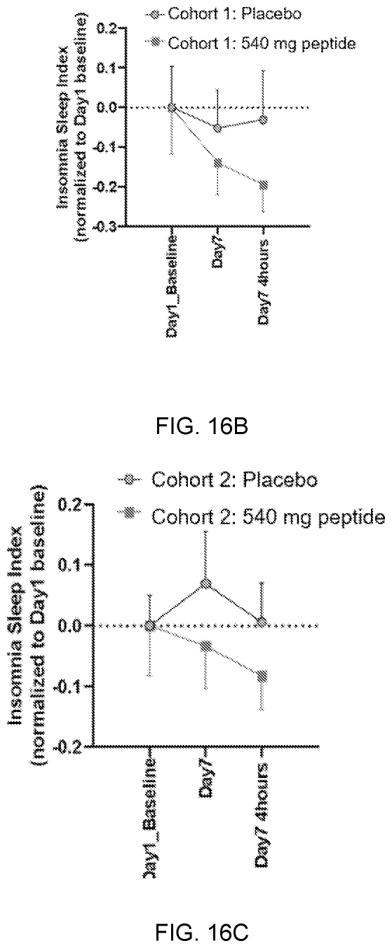

Sleep quality was also assessed in the MAD cohorts using the insomnia severity index (ISI), a multidimensional 7-item self-reported scale that evaluates the severity of sleep quality issues (0=no problem to 4=severe problem). In MAD Cohort 1, dosing for 7 days with the peptide resulted in a dose-dependent improvement in sleep quality as evidenced by a lower ISI. (See FIG. 16A). Improved sleep quality was observed after 7 days of dosing with 540 mg of the peptide in both MAD Cohort 1 (FIG. 16B) and in the crossover study (MAD Cohort 2) (FIG. 16C.)

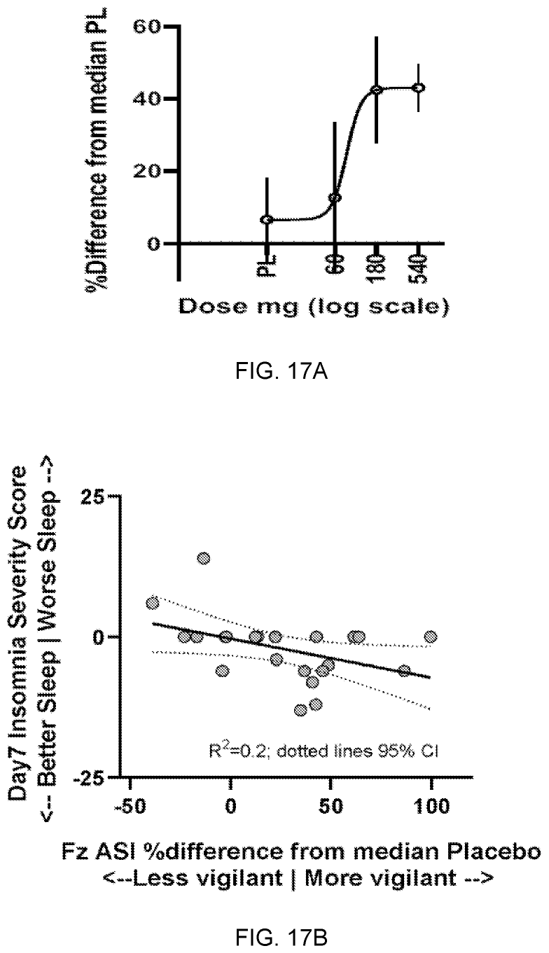

The alpha slow wave index (ASI; alpha/(delta+theta)) in the frontal cortex is widely used as an objective measure of vigilance. Increases in ASI indicate vigilance, whereas decreases in ASI indicate sleepiness or lack of focus. As shown in FIG. 17A, the peptide-treated subjects exhibited a dose-dependent increase in the ASI. Improvements in reported sleep quality correlated with ASI changes in the frontal cortex (Fz eyes closed). See FOG. 17B.

The results suggest that the peptide LSSTQAQQSY (SEQ ID NO: 1) modulates Alpha power in eyes open and closed states. Whereas Alpha power is reduced in Alzheimer's, Parkinson's, and cognitive impairment, the peptide LSSTQAQQSY (SEQ ID NO: 1) increases in Alpha power, and this increase is correlated with improvements in cognition, mood, and sleepiness.

### Example 5: Pharmacological Study with Felypressin

The effect of orally administered felypressin, a selective AVPR1A agonist, on antidepressant activity in the mouse tail suspension test was evaluated. Felypressin at 0.03 mg/kg, 0.1 mg/kg, and 0.3 mg/kg was compared to the peptide LSSTQAQQSY (SEQ ID NO: 1) at 0.3 mg/kg (n=6 per group). The effect of orally administered felypressin (0.1 mg/kg) was also compared to the effect of orally administered felypressin (0.1 mg/kg) plus the AVPR1A/AVPR2 inhibitor conivaptan (3 mg/kg) (n=7 per group).

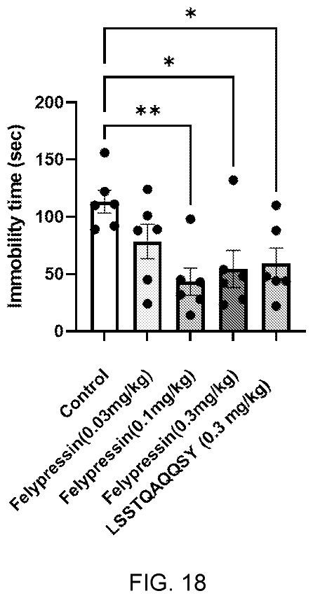

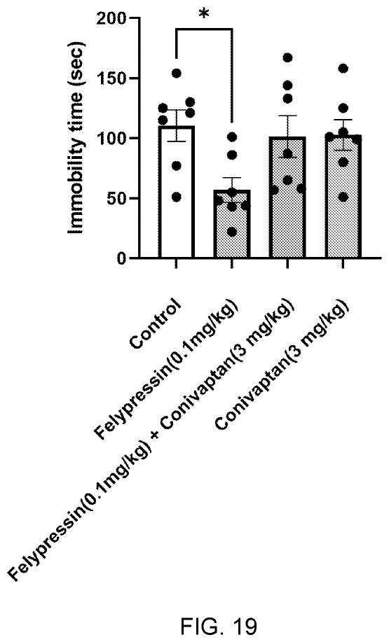

Results are shown in FIG. 18 and FIG. 19. As shown in FIG. 18 and FIG. 19, AVPR1A activation induces an antidepressant effect. The AVPR1A/AVPR2 inhibitor conivaptan blocks felypressin activity in the tail suspension test.

### Example 6: Pharmacological Study with Desmopressin

The effect of orally administered desmopressin, which is exhibits greater selectivity for AVPR2 than AVPR1A, on antidepressant activity in the mouse tail suspension test was evaluated. Desmopressin at 0.03 mg/kg, 0.1 mg/kg, and 0.3 mg/kg was compared to felypressin at 0.1 mg/kg (n=6 per group) in a first study. Desmopressin at 0.1 mg/kg, 0.3 mg/kg, and 1.0 mg/kg was compared to felypressin at 0.1 mg/kg (n=6 per group) in a first study.

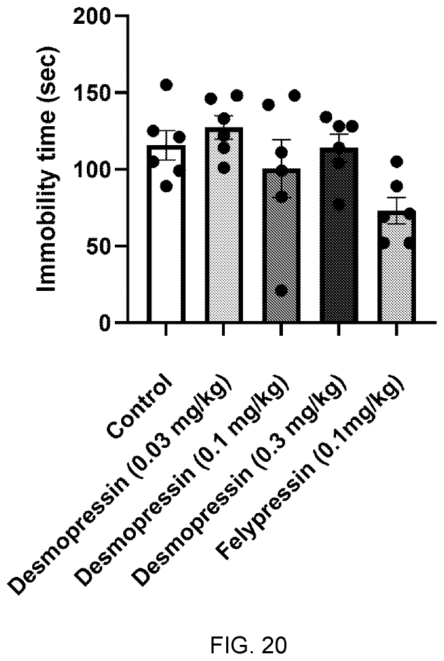

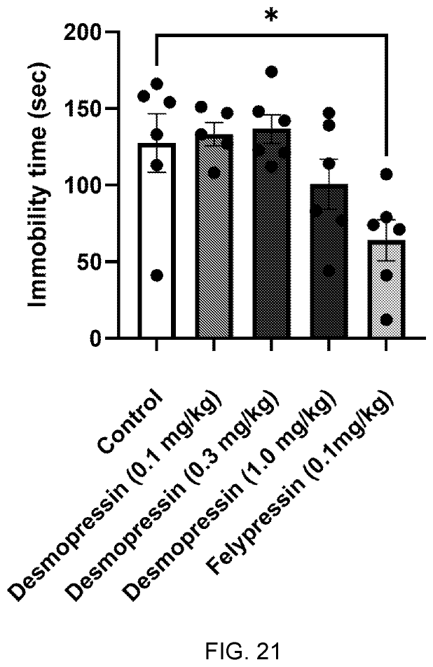

Results are shown in FIG. 20 and FIG. 21. As shown in FIG. 20 and FIG. 21, desmopressin showed less of an effect than felypressin.

### Example 7: Histological Analysis of Peptide LSSTQAQQSY (SEQ ID NO: 1) in the Upper Gut

To understand the mechanism of action for peptide LSSTQAQQSY (SEQ ID NO: 1), tissue sections from mouse gastrointestinal tissue were stained with fluorescently labeled peptide and with cell-specific markers using different colors for each stain. Peptide binding did not overlap with a general enterocyte marker staining; however peptide binding overlapped with an enteroendocrine sensory cell marker. (Data not shown.)

These results indicate that the peptide interacts with sensory cells in the upper gut.

### Example 8: Dose Dependency and Effect of Repeat Dosing of Peptide LSSTQAQQSY (SEQ ID NO: 1)

Oral administration of the peptide LSSTQAQQSY (SEQ ID NO: 1) promotes stress resilience in mice, as evidence in a tail suspension test (TST). The present Example examines the dose dependency of this effect and whether repeat dosing affects efficacy.

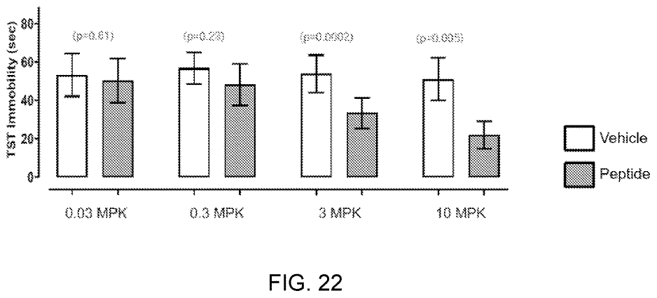

Mice were administered either vehicle or peptide at 0.03 mg/kg, 0.3 mg/kg, 3 mg/kg, or 10 mg/kg of the peptide and were subject to the TST. Reduced immobility time (indicative of stress resilience) was observed at 3 mg/kg and 10 mg/kg, with a greater difference between vehicle and peptide-treated groups in the 10 mg/kg dose. (See FIG. 22)

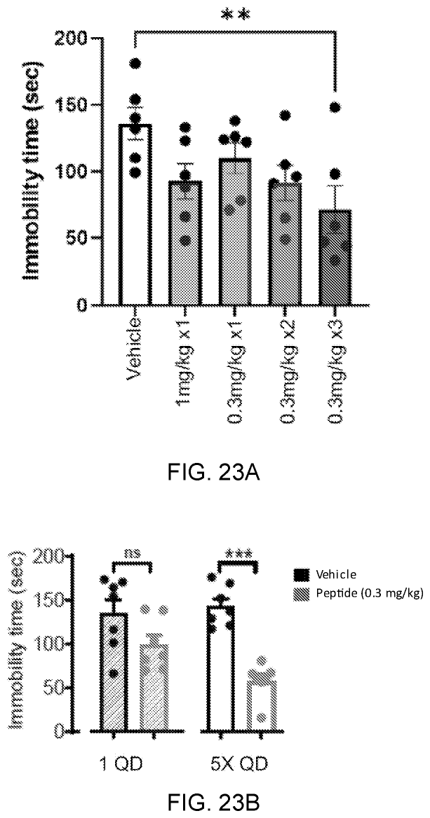

In another series of experiments, mice were dosed repeatedly with the peptide and subjected to the TST. Repeat dosing with the peptide at two hour intervals (FIG. 23A) and at daily for 5 days (FIG. 23B) both increased efficacy.

### CITATION OF REFERENCES

While various specific embodiments have been illustrated and described, it will be appreciated that various changes can be made without departing from the spirit and scope of the disclosure(s).

### NUMBERED EMBODIMENTS

The present disclosure is exemplified by the numbered embodiments set forth below.

1. A method of (a) treating a subject suffering from or at risk of (i) a disease or disorder of the nervous system or (ii) an inflammatory disease or disorder, or (b) improving sleep in a subject or (c) improving cognition in a subject, comprising administering to the subject a therapeutically effective amount of an arginine vasopressin receptor 1A (AVPR1A) agonist, optionally wherein the AVPR1 agonist is an AVPR1A agonist described herein.

2. The method of embodiment 1, wherein the AVPR1A agonist has greater selectivity for AVPR1A than AVPR1B.

3. The method of embodiment 1 or embodiment 2, wherein the AVPR1A agonist has greater selectivity for AVPR1A than AVPR2.

4. The method of embodiment 1, wherein the AVPR1A agonist is vasopressin, a vasopressin analog, or a pharmaceutically acceptable salt thereof.

5. The method of embodiment 1 or embodiment 4, wherein the AVPR1A agonist is vasopressin or a pharmaceutically acceptable salt thereof.

6. The method of embodiment 1 or embodiment 4, wherein the AVPR1A agonist is a vasopressin analog or a pharmaceutically acceptable salt thereof.

7. The embodiment 6, wherein the vasopressin analog has greater selectivity for AVPR1A than AVPR1B.

8. The method of embodiment 6 or embodiment 7, wherein the AVPR1A agonist has greater selectivity for AVPR1A than AVPR2.

9. The method of embodiment 1 or embodiment 6, wherein the AVPR1A agonist is desmopressin, felypressin, ornipressin, selepressin, terlipressin, lypressin, F180, [deamino-Cys(1)]arginine vasopressin (dAVP), or a pharmaceutically acceptable salt thereof.

10. The method of embodiment 9, wherein the AVPR1A agonist is desmopressin or a pharmaceutically acceptable salt thereof.

11. The method of embodiment 9, wherein the AVPR1A agonist is felypressin or a pharmaceutically acceptable salt thereof.

12. The method of embodiment 9, wherein the AVPR1A agonist is ornipressin or a pharmaceutically acceptable salt thereof.

13. The method of embodiment 9, wherein the AVPR1A agonist is selepressin or a pharmaceutically acceptable salt thereof.

14. The method of embodiment 9, wherein the AVPR1A agonist is terlipressin or a pharmaceutically acceptable salt thereof.

15. The method of embodiment 9, wherein the AVPR1A agonist is lypressin or a pharmaceutically acceptable salt thereof.

16. The method of embodiment 9, wherein the AVPR1A agonist is F180 or a pharmaceutically acceptable salt thereof.

17. The method of embodiment 9, wherein the AVPR1A agonist is [deamino-Cys(1)]arginine vasopressin (dAVP) or a pharmaceutically acceptable salt thereof.

18. The method of any one of embodiments 1 to 17, wherein the subject is suffering from or at risk of a disease or disorder of the nervous system.

19. The method of embodiment 18, wherein the subject is suffering from or at risk of a disease or disorder of the central nervous system.

20. The method of any one of embodiments 1 to 17, wherein the subject is suffering from or at risk of an inflammatory disease or disorder.

21. The method of any one of embodiments 1 to 17, which is a method of improving sleep in a subject.

22. The method of any one of embodiments 1 to 17, wherein the subject is suffering from or at risk of an inflammatory disease or disorder, schizophrenia or psychosis, a neurodegenerative disease, a gastrointestinal disease or disorder, pain, rheumatoid arthritis, migraine, headache, substance abuse, drug addiction, a seizure disorder, major depressive disorder, atypical depression, major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, or a sleep disorder.

23. The method of any one of embodiments 1 to 17, wherein the subject is suffering from or at risk of schizophrenia or psychosis.

24. The method of embodiment 23, wherein the subject is suffering from or at risk of schizophrenia.

25. The method of any one of embodiments 23 to 24, wherein the subject exhibits psychotic behavior.

26. The method of any one of embodiments 24 to 26, wherein the treating comprises improving or slowing progression of one or more negative symptoms of schizophrenia or psychosis.

27. The method of any one of embodiments 1 to 26, wherein the treating comprises improving or slowing progression of one or more negative symptoms of a disease or disorder of the central nervous system.

28. The method of embodiment 26 or embodiment 27, wherein the one or more negative symptoms comprise asociality, anhedonia, alogia, affective flattening, apathy, avolition, blunted affect, anergia, apathy, depression, low mood, cognitive impairment or a combination thereof.

29. The method of embodiment 28, wherein the one or more negative symptoms comprise asociality.

30. The method of embodiment 28 or embodiment 29, wherein the one or more negative symptoms comprise anhedonia.

31. The method of any one of embodiments 28 to 30, wherein the one or more negative symptoms comprise alogia.

32. The method of any one of embodiments 28 to 31, wherein the one or more negative symptoms comprise affective flattening.

33. The method of any one of embodiments 28 to 32, wherein the one or more negative symptoms comprise apathy.

34. The method of any one of embodiments 28 to 33, wherein the one or more negative symptoms comprise avolition.

35. The method of any one of embodiments 28 to 34, wherein the one or more negative symptoms comprise blunted affect.

36. The method of any one of embodiments 28 to 35, wherein the one or more negative symptoms comprise anergia.

37. The method of any one of embodiments 28 to 36, wherein the one or more negative symptoms comprise apathy.

38. The method of any one of embodiments 28 to 37, wherein the one or more negative symptoms comprise depression.

39. The method of any one of embodiments 28 to 38, wherein the one or more negative symptoms comprise low mood.

40. The method of any one of embodiments 28 to 39, wherein the one or more negative symptoms comprise cognitive impairment.

41. The method of any one of embodiments 1 to 40, wherein the treating comprises improving or slowing progression of a cognitive deficit in the subject, optionally wherein the cognitive deficit comprises a deficit in verbal working memory, spatial working memory, verbal fluency, verbal learning, or a combination thereof.

42. The method of any one of embodiments 1 to 41, wherein the subject is suffering from a neurodegenerative disease.

43. The method of any one of embodiments 1 to 42, wherein the subject is suffering from Parkinson's disease.

44. The method of embodiment 43, wherein the treating comprises improving or slowing progression of one or more non-motor symptoms of the subject's Parkinson's disease.

45. The method of embodiment 44, wherein the one or more non-motor symptoms comprise a sensory symptom, a cognitive symptom, autonomic symptom, or a combination thereof.

46. The method of embodiment 45, wherein the one or more non-motor symptoms comprise one or more sensory symptoms.

47. The method of embodiment 46, wherein the one or more sensory symptoms comprise numbness, restlessness, pain, chest discomfort, anosmia, or a combination thereof.

48. The method of any one of embodiments 44 to 47, wherein the one or more non-motor symptoms comprise one or more cognitive symptoms.

49. The method of embodiment 48, wherein the one or more cognitive symptoms comprise mood changes, depression, anxiety, panic attacks, tiredness, confusion, slowed thinking, or a combination thereof.

50. The method of any one of embodiments 45 to 49, wherein the one or more non-motor symptoms comprise one or more autonomic symptoms.

51. The method of embodiment 50, wherein the one or more autonomic symptoms comprise hot/cold sensations, bladder problems, sweating, abdominal discomfort, constipation, sialorrhea, frequent urination and/or urgency, erectile dysfunction, or a combination thereof.

52. The method of any one of embodiments 44 to 51, wherein the one or more non-motor symptoms comprise cognition deficit and/or impairment, depression, anxiety, fatigue, apathy, or a combination thereof.

53. The method of any one of embodiments 1 to 17, wherein the subject is suffering from a gastrointestinal disease or disorder.

54. The method of any one of embodiments 1 to 17, wherein the subject is suffering from irritable bowel syndrome.

55. The method of any one of embodiments 1 to 17, wherein the subject is suffering from inflammatory bowel disease.

56. The method of any one of embodiments 1 to 17, wherein the subject is suffering from Crohn's disease.

57. The method of any one of embodiments 1 to 17, wherein the subject is suffering from ulcerative colitis.

58. The method of any one of embodiments 1 to 17, wherein the subject is suffering from pain (e.g., visceral pain).

59. The method of any one of embodiments 1 to 17, wherein the subject is suffering from rheumatoid arthritis.

60. The method of any one of embodiments 1 to 17, wherein the subject is suffering from a migraine.

61. The method of any one of embodiments 1 to 17, wherein the subject is suffering from a headache.

62. The method of any one of embodiments 1 to 17, wherein the subject is suffering from substance abuse.

63. The method of any one of embodiments 1 to 17, wherein the subject is suffering from drug addiction.

64. The method of any one of embodiments 1 to 17, wherein the subject is suffering from a seizure disorder.

65. The method of embodiment 64, wherein the seizure disorder is epilepsy.

66. The method of embodiment 65, wherein the epilepsy is an orphan epilepsy.

67. The method of any one of embodiments 1 to 17, wherein the subject is suffering from major depressive disorder.

68. The method of any one of embodiments 1 to 17, wherein the subject is suffering from atypical depression.

69. The method of any one of embodiments 1 to 17, wherein the subject is suffering from a major depressive episode (MDE) (e.g., atypical MDE).

70. The method of any one of embodiments 1 to 17, wherein the subject is suffering from treatment resistant depression.

71. The method of any one of embodiments 1 to 17, wherein the subject is suffering from depression in the presence of a neurodegenerative disease (e.g., Parkinson's disease or Alzheimer's disease).

72. The method of any one of embodiments 1 to 17, wherein the subject is suffering from cognitive impairment.

73. The method of embodiment 72, wherein the cognitive impairment is minor cognitive impairment.

74. The method of any one of embodiments 1 to 17, wherein the subject is suffering from COVID-19 related cognitive impairment and/or depression.

75. The method of any one of embodiments 1 to 17, wherein the subject is suffering from Alzheimer's disease.

76. The method of any one of embodiments 1 to 17, wherein the subject is suffering from ADHD.

77. The method of any one of embodiments 1 to 17, wherein the subject is suffering from an autism spectrum disorder.

78. The method of any one of embodiments 1 to 17, wherein the subject is suffering from a pervasive developmental disorder, which is optionally Asperger syndrome or Rett syndrome.

79. The method of any one of embodiments 1 to 17, wherein the subject is suffering from atypical autism.

80. The method of any one of embodiments 1 to 17, wherein the subject is suffering from multiple sclerosis.

81. The method of any one of embodiments 1 to 17, wherein the subject is suffering from PTSD.

82. The method of any one of embodiments 1 to 17, wherein the subject is suffering from a sleep disorder.

83. The method of any one of embodiments 1 to 17, wherein the subject is suffering from insomnia.

84. The method of any one of embodiments 1 to 17, wherein the subject is suffering from daytime fatigue.

85. The method of any one of embodiments 1 to 17, wherein the subject is suffering from REM sleep behavior disorder.

86. The method of any one of embodiments 1 to 17, wherein the treating comprises improving sleep in a subject.

87. The method of any one of embodiments 1 to 17, wherein the subject is suffering from or at risk of a mental disorder.

88. The method of embodiment 87, wherein the mental disorder is a mood disorder, an anxiety disorder, or a disorder of diminished motivation.

89. The method of embodiment 88, wherein the mental disorder is a mood disorder.

90. The method of embodiment 89, wherein the mood disorder comprises depression, bipolar disorder, or adjustment disorder.

91. The method of any one of embodiments 87 to 90, wherein the mental disorder comprises depression.

92. The method of any one of embodiments 87 to 90, wherein the mental disorder bipolar disorder.

93. The method of any one of embodiments 87 to 90, wherein the mental disorder comprises adjustment disorder.

94. The method of embodiment 88, wherein the mental disorder is an anxiety disorder.

95. The method of embodiment 88, wherein the mental disorder is a disorder of diminished motivation.

96. The method of embodiment 95, wherein the disorder of diminished motivation comprises apathy, abulia, or akinetic mutism.

97. The method of embodiment 96, wherein the disorder of diminished motivation comprises apathy.

98. The method of embodiment 96, wherein the disorder of diminished motivation comprises abulia.

99. The method of embodiment 96, wherein the disorder of diminished motivation comprises akinetic mutism.

100. The method of any one of embodiments 1 to 99, wherein the AVPR1A agonist is administered as monotherapy.

101. The method of any one of embodiments 1 to 100, wherein the AVPR1A agonist is administered orally.

102. The method of any one of embodiments 1 to 101, wherein the subject has an elevated TNF-α level.

103. An AVPR1A agonist for use in a method as described in any one of embodiments 1 to 102.

104. Use of an AVPR1A agonist in the manufacture of a medicament for treating any disease or disorder described in any one of embodiments 1 to 102.

### 1. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1. A method of (a) treating a subject suffering from or at risk of (i) a disease or disorder of the nervous system or (ii) an inflammatory disease or disorder, or (b) improving sleep in a subject or (c) improving cognition in a subject, comprising administering to the subject a therapeutically effective amount of an arginine vasopressin receptor 1A (AVPR1A) agonist, optionally wherein the AVPR1 agonist is an AVPR1A agonist described herein.

2. A method of (a) treating a subject suffering from or at risk of (i) a disease or disorder of the nervous system or (ii) an inflammatory disease or disorder, or (b) improving sleep in a subject or (c) improving cognition in a subject, comprising administering to the subject a therapeutically effective amount of an agent comprising a means for agonizing arginine vasopressin receptor 1A (AVPR1A).

3. The method of claim 2, wherein the means for agonizing comprises an AVPR1A agonist.

4. The method of claim 1 or 3, wherein the AVPR1A agonist is vasopressin, a vasopressin analog, or a pharmaceutically acceptable salt thereof.

5. The method of claim 1, 3, or 4, wherein the AVPR1A agonist is vasopressin or a pharmaceutically acceptable salt thereof.

6. The method of claim 1, 3, or 4, wherein the AVPR1A agonist is a vasopressin analog or a pharmaceutically acceptable salt thereof.

7. The method of claim 1 or claim 6, wherein the AVPR1A agonist is felypressin, desmopressin, ornipressin, selepressin, terlipressin, lypressin, F180, [deamino-Cys(1)]arginine vasopressin (dAVP), or a pharmaceutically acceptable salt thereof.

8. The method of any one of claims 1 to 7, wherein the subject is suffering from or at risk of a disease or disorder of the nervous system.

9. The method of claim 8, wherein the subject is suffering from or at risk of a disease or disorder of the central nervous system.

10. The method of any one of claims 1 to 7, wherein the subject is suffering from or at risk of an inflammatory disease or disorder.

11. The method of any one of claims 1 to 7, which is a method of improving sleep in a subject.

12. The method of any one of claims 1 to 7, wherein the subject is suffering from or at risk of an inflammatory disease or disorder, schizophrenia or psychosis, a neurodegenerative disease, a gastrointestinal disease or disorder, pain, rheumatoid arthritis, migraine, headache, substance abuse, drug addiction, a seizure disorder, major depressive disorder, atypical depression, major depressive episode (MDE), treatment resistant depression, depression in the presence of a neurodegenerative disease, cognitive impairment, COVID-19 related cognitive impairment and/or depression, ADHD, an autism spectrum disorder, a pervasive developmental disorder, atypical autism, multiple sclerosis, PTSD, or a sleep disorder.

13. The method of any one of claims 1 to 7, wherein the subject is suffering from or at risk of schizophrenia or psychosis.

14. The method of claim 13, wherein the subject is suffering from or at risk of schizophrenia.

15. The method of claims 13 or 14, wherein the subject exhibits psychotic behavior.

16. The method of any one of claims 14 to 16, wherein the treating comprises improving or slowing progression of one or more negative symptoms of schizophrenia or psychosis.

17. The method of any one of claims 1 to 17, wherein the treating comprises improving or slowing progression of one or more negative symptoms of a disease or disorder of the central nervous system.

18. The method of claim 16 or claim 17, wherein the one or more negative symptoms comprise asociality, anhedonia, alogia, affective flattening, apathy, avolition, blunted affect, anergia, apathy, depression, low mood, cognitive impairment or a combination thereof.

19. The method of claim 18, wherein the one or more negative symptoms comprise asociality.

20. The method of claim 18 or claim 19, wherein the one or more negative symptoms comprise anhedonia.

21. The method of any one of claims 18 to 20, wherein the one or more negative symptoms comprise alogia.

22. The method of any one of claims 18 to 21, wherein the one or more negative symptoms comprise affective flattening.

23. The method of any one of claims 18 to 22, wherein the one or more negative symptoms comprise apathy.

24. The method of any one of claims 18 to 23, wherein the one or more negative symptoms comprise avolition.

25. The method of any one of claims 18 to 24, wherein the one or more negative symptoms comprise blunted affect.

26. The method of any one of claims 18 to 25, wherein the one or more negative symptoms comprise anergia.

27. The method of any one of claims 18 to 26, wherein the one or more negative symptoms comprise apathy.

28. The method of any one of claims 18 to 27, wherein the one or more negative symptoms comprise depression.

29. The method of any one of claims 18 to 28, wherein the one or more negative symptoms comprise low mood.

30. The method of any one of claims 18 to 29, wherein the one or more negative symptoms comprise cognitive impairment.

31. The method of any one of claims 1 to 30, wherein the treating comprises improving or slowing progression of a cognitive deficit in the subject, optionally wherein the cognitive deficit comprises a deficit in verbal working memory, spatial working memory, verbal fluency, verbal learning, or a combination thereof.

32. The method of any one of claims 1 to 31, wherein the subject is suffering from a neurodegenerative disease.

33. The method of any one of claims 1 to 32, wherein the subject is suffering from Parkinson's disease.

34. The method of claim 33, wherein the treating comprises improving or slowing progression of one or more non-motor symptoms of the subject's Parkinson's disease.

35. The method of claim 34, wherein the one or more non-motor symptoms comprise a sensory symptom, a cognitive symptom, autonomic symptom, or a combination thereof.

36. The method of claim 35, wherein the one or more non-motor symptoms comprise one or more sensory symptoms.

37. The method of claim 36, wherein the one or more sensory symptoms comprise numbness, restlessness, pain, chest discomfort, anosmia, or a combination thereof.

38. The method of any one of claims 34 to 37, wherein the one or more non-motor symptoms comprise one or more cognitive symptoms.

39. The method of claim 38, wherein the one or more cognitive symptoms comprise mood changes, depression, anxiety, panic attacks, tiredness, confusion, slowed thinking, or a combination thereof.

40. The method of any one of claims 35 to 39, wherein the one or more non-motor symptoms comprise one or more autonomic symptoms.

41. The method of claim 40, wherein the one or more autonomic symptoms comprise hot/cold sensations, bladder problems, sweating, abdominal discomfort, constipation, sialorrhea, frequent urination and/or urgency, erectile dysfunction, or a combination thereof.

42. The method of any one of claims 34 to 41, wherein the one or more non-motor symptoms comprise cognition deficit and/or impairment, depression, anxiety, fatigue, apathy, or a combination thereof.

43. The method of any one of claims 1 to 7, wherein the subject is suffering from a gastrointestinal disease or disorder.

44. The method of any one of claims 1 to 7, wherein the subject is suffering from irritable bowel syndrome.

45. The method of any one of claims 1 to 7, wherein the subject is suffering from inflammatory bowel disease.

46. The method of any one of claims 1 to 7, wherein the subject is suffering from Crohn's disease.

47. The method of any one of claims 1 to 7, wherein the subject is suffering from ulcerative colitis.

48. The method of any one of claims 1 to 7, wherein the subject is suffering from pain (e.g., visceral pain).

49. The method of any one of claims 1 to 7, wherein the subject is suffering from rheumatoid arthritis.

50. The method of any one of claims 1 to 7, wherein the subject is suffering from a migraine.

51. The method of any one of claims 1 to 7, wherein the subject is suffering from a headache.

52. The method of any one of claims 1 to 7, wherein the subject is suffering from substance abuse.

53. The method of any one of claims 1 to 7, wherein the subject is suffering from drug addiction.

54. The method of any one of claims 1 to 7, wherein the subject is suffering from a seizure disorder.

55. The method of claim 54, wherein the seizure disorder is epilepsy.

56. The method of claim 55, wherein the epilepsy is an orphan epilepsy.

57. The method of any one of claims 1 to 7, wherein the subject is suffering from major depressive disorder.

58. The method of any one of claims 1 to 7, wherein the subject is suffering from atypical depression.

59. The method of any one of claims 1 to 7, wherein the subject is suffering from a major depressive episode (MDE) (e.g., atypical MDE).

60. The method of any one of claims 1 to 7, wherein the subject is suffering from treatment resistant depression.

61. The method of any one of claims 1 to 7, wherein the subject is suffering from depression in the presence of a neurodegenerative disease (e.g., Parkinson's disease or Alzheimer's disease).

62. The method of any one of claims 1 to 7, wherein the subject is suffering from cognitive impairment.

63. The method of claim 62, wherein the cognitive impairment is minor cognitive impairment.

64. The method of any one of claims 1 to 7, wherein the subject is suffering from COVID-19 related cognitive impairment and/or depression.

65. The method of any one of claims 1 to 7, wherein the subject is suffering from Alzheimer's disease.

66. The method of any one of claims 1 to 7, wherein the subject is suffering from ADHD.

67. The method of any one of claims 1 to 7, wherein the subject is suffering from an autism spectrum disorder.

68. The method of any one of claims 1 to 7, wherein the subject is suffering from a pervasive developmental disorder, which is optionally Asperger syndrome or Rett syndrome.

69. The method of any one of claims 1 to 7, wherein the subject is suffering from atypical autism.

70. The method of any one of claims 1 to 7, wherein the subject is suffering from multiple sclerosis.

71. The method of any one of claims 1 to 7, wherein the subject is suffering from PTSD.

72. The method of any one of claims 1 to 7, wherein the subject is suffering from a sleep disorder.

73. The method of any one of claims 1 to 7, wherein the subject is suffering from insomnia.

74. The method of any one of claims 1 to 7, wherein the subject is suffering from daytime fatigue.

75. The method of any one of claims 1 to 7, wherein the subject is suffering from REM sleep behavior disorder.

76. The method of any one of claims 1 to 7, wherein the treating comprises improving sleep in a subject.

77. The method of any one of claims 1 to 7, wherein the subject is suffering from or at risk of a mental disorder.

78. The method of claim 77, wherein the mental disorder is a mood disorder, an anxiety disorder, or a disorder of diminished motivation.

79. The method of claim 78, wherein the mental disorder is a mood disorder.

80. The method of claim 79, wherein the mood disorder comprises depression, bipolar disorder, or adjustment disorder.

81. The method of any one of claims 77 to 80, wherein the mental disorder comprises depression.

82. The method of any one of claims 77 to 80, wherein the mental disorder bipolar disorder.

83. The method of any one of claims 77 to 80, wherein the mental disorder comprises adjustment disorder.

84. The method of claim 78, wherein the mental disorder is an anxiety disorder.

85. The method of claim 78, wherein the mental disorder is a disorder of diminished motivation.

86. The method of claim 85, wherein the disorder of diminished motivation comprises apathy, abulia, or akinetic mutism.

87. The method of claim 86, wherein the disorder of diminished motivation comprises apathy.

88. The method of claim 86, wherein the disorder of diminished motivation comprises abulia.

89. The method of claim 86, wherein the disorder of diminished motivation comprises akinetic mutism.

90. The method of any one of claims 1 to 89, wherein the AVPR1A agonist is administered as monotherapy.

91. The method of any one of claims 1 to 90, wherein the AVPR1A agonist is administered orally.

92. The method of any one of claims 1 to 91, wherein the subject has an elevated TNF-α level.

93. A method of treating a subject suffering from or at risk of Alzheimer's Disease or Parkinson's Disease comprising administering to the subject a therapeutically effective amount of a vasopressin analog or a pharmaceutically acceptable salt thereof.

94. A method of improving cognition in a subject comprising administering to the subject a therapeutically effective amount of a vasopressin analog or a pharmaceutically acceptable salt thereof.

95. A method of improving sleep in a subject comprising administering to the subject a therapeutically effective amount of a vasopressin analog or a pharmaceutically acceptable salt thereof.

96. A method of treating a subject suffering from or at risk of depression in a subject comprising administering to the subject a therapeutically effective amount of a vasopressin analog or a pharmaceutically acceptable salt thereof.

97. The method of any one of claims 93-96, wherein the subject is a mammal.

98. The method of claim 97, wherein the subject is human.

99. The method of any one of claims 93-98, wherein the vasopressin analog is felypressin or desmopressin.

100. The method of any one of claims 1-99, comprising administering the AVPR1A agonist or agent in multiple doses.

101. An AVPR1A agonist for use in a method as described in any one of claims 1 to 100.

102. Use of an AVPR1A agonist in the manufacture of a medicament for treating any disease or disorder described herein.


SEQUENCE LISTING (1 sequence)

SEQ ID NO: 1 - protein, 10 aa, Artificial sequence
LSSTQAQQSY